(19) United States
(12) Patent Application Publication
(10) Pub. No.: US 2024/0124514 A1
(43) Pub. Date: Apr. 18, 2024

(54) DEOXYNUCLEOSIDE MODIFIED RUTHENIUM COMPLEX, AND PREPARATION METHOD AND USE THEREOF

(71) Applicant: Guangzhou Ruby Biotechnology Co., Ltd., Guangdong (CN)

(72) Inventors: Shuyuan ZHOU, Guangdong (CN); Qiong WU, Guangdong (CN)

(73) Assignee: Guangzhou Ruby Biotechnology Co., Ltd., Guangdong (CN)

(21) Appl. No.: 18/458,250

(22) Filed: Aug. 30, 2023

(30) Foreign Application Priority Data
Sep. 26, 2022 (CN) 202211172829.0

(51) Int. Cl. C07H 23/00

(52) U.S. Cl. CPC C07H 23/00

(57) ABSTRACT

Provided are a deoxynucleoside modified ruthenium complex, and a preparation method and use thereof, The deoxynucleoside modified ruthenium complex has a structure represented by Formula I, wherein R represents one selected from the group consisting of trifluoromethyl, C₁₋₆ alkyl, alkoxy, halogen, styryl, and nitro-substituted styryl.

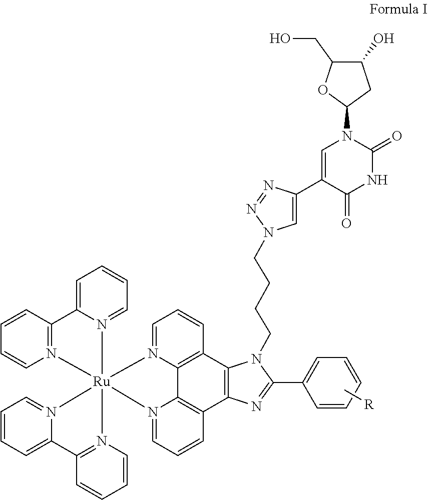

### CROSS REFERENCE TO RELATED APPLICATION

This patent application claims the priority of Chinese Patent Application No. 202211172829.0, entitled “Deoxynucleoside modified ruthenium complex, and preparation method and use thereof” filed with the China National Intellectual Property Administration on Sep. 26, 2022, the disclosure of which is incorporated herein by reference in its entirety.

### TECHNICAL FIELD

The present disclosure relates to the field of medicinal chemistry, and specifically relates to a deoxynucleoside modified ruthenium complex, and a preparation method and use thereof.

### BACKGROUND

COVID-19 (Corona Virus Disease 2019) is caused by the coronavirus SARS-CoV-2, which has mutated many times since its discovery, and has spread widely and become a worldwide pandemic disease. At present, most of the measures to deal with COVID-19 are limited to vaccination prevention and post-infection symptomatic treatment. There is still no specific medicine for the etiological treatment of COVID-19.

G-quadruplex is a high-level structure formed by folding DNA or RNA rich in tandemly repeated guanines (G). G-quartet is a structural unit of the G-quadruplex, and is a ring plane formed by four Gs that are connected by Hoogsteen hydrogen bond. The quadruplex is formed by π-π stacking two or more layers of quartets. Qu Xiaogang et al. have predicted and verified the existence of G4 RNA sequence in the sequence of novel coronavirus, and found that the translation process of the virus could be effectively prevented by adding a G4 RNA binding stabilizer to interact with G4 RNA, indicating that G4 RNA can be a drug action target of anti-novel coronavirus.

### SUMMARY

In view of the above, an object of the present disclosure is to provide a deoxynucleoside modified ruthenium complex, and a preparation method and use thereof. In the present disclosure, the deoxynucleoside modified ruthenium complex could well target, recognize and bind with G4 RNA, especially G4 RNA in novel coronavirus. Therefore, the deoxynucleoside modified ruthenium complex exhibits a potential effect of antiviral, especially anti-novel coronavirus and is expected to develop into an anti-novel coronavirus drug.

In order to achieve the above object, the present disclosure provides the following technical solutions:

The present disclosure provides a deoxynucleoside modified ruthenium complex, having a structure represented by Formula I:

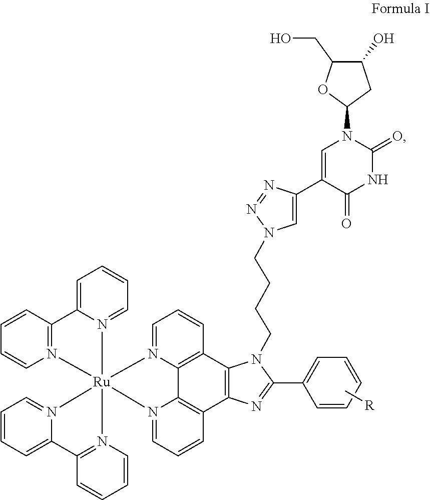

wherein R represents one selected from the group consisting of trifluoromethyl, C₁₋₆ alkyl, alkoxy, halogen, styryl, and nitro-substituted styryl.

In some embodiments, the deoxynucleoside modified ruthenium complex has a structure represented by any one of Formulas 1 to 10:

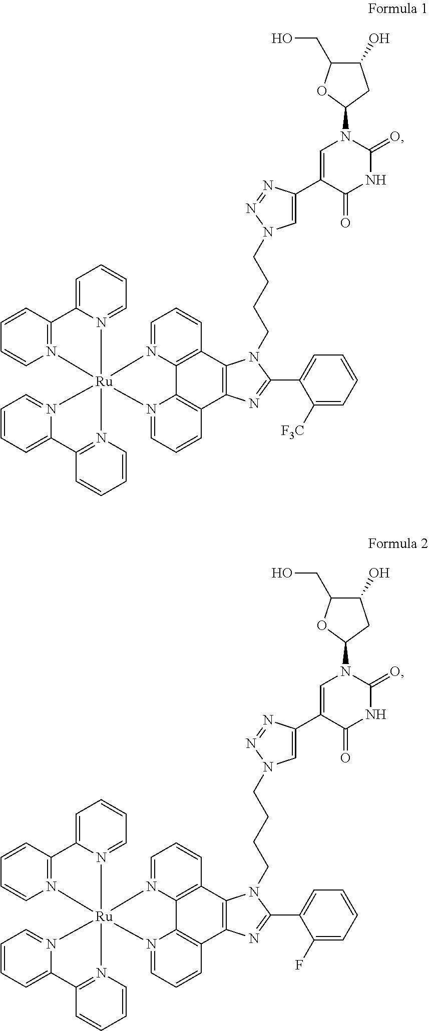

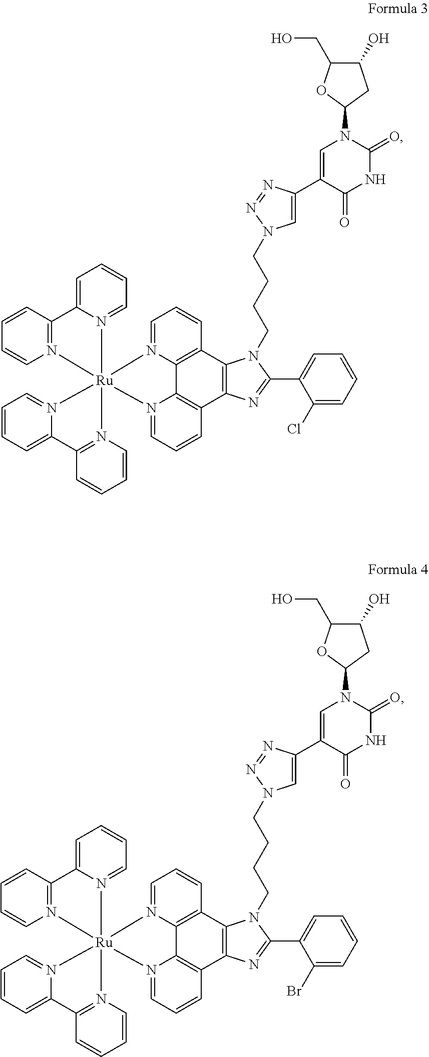

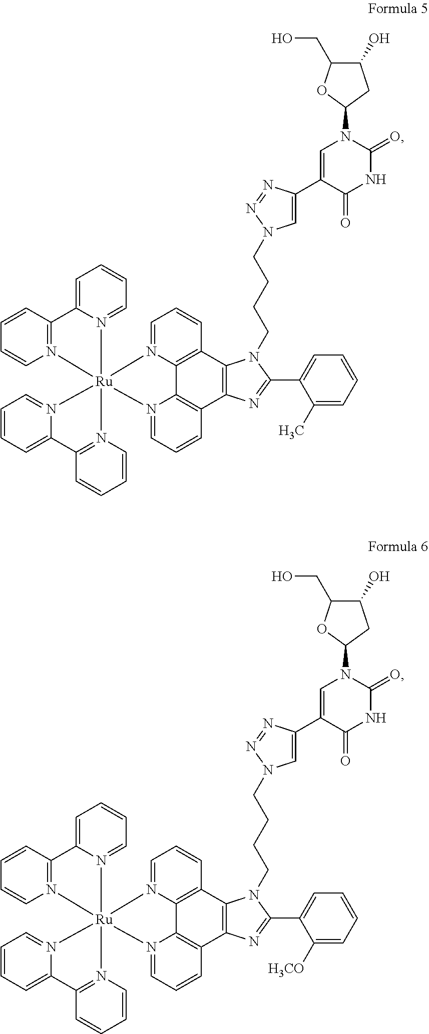

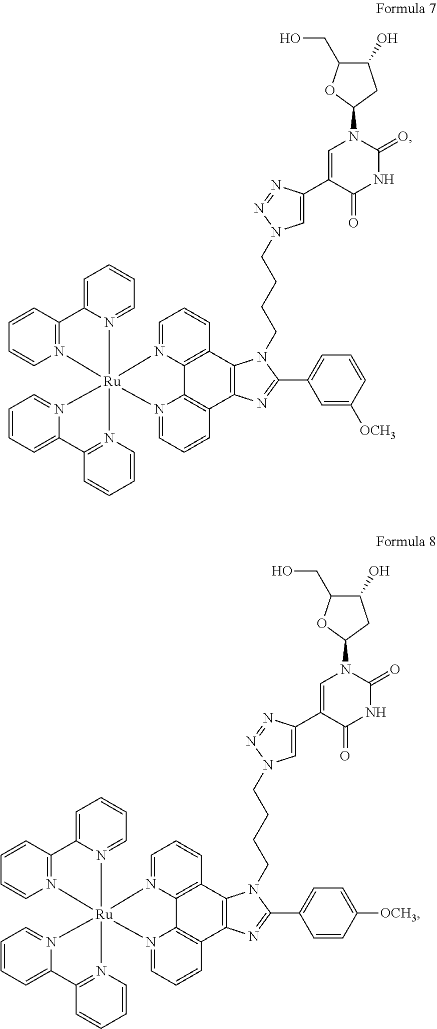

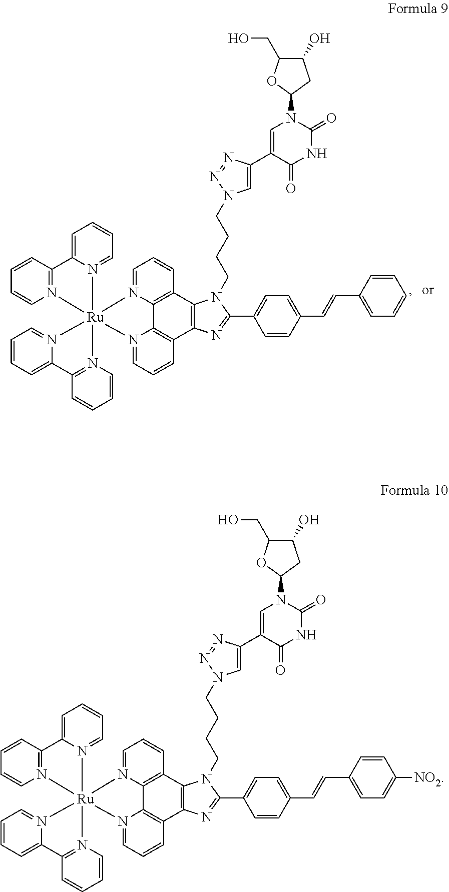

The present disclosure further provides a method for preparing the deoxynucleoside modified ruthenium complex described in the above technical solutions, comprising:

mixing a ruthenium complex azide, 5-ethynyl-2′-deoxyuridine, copper sulfate, sodium ascorbate, and a solvent to obtain a mixture, and subjecting the mixture to microwave heating radiation under a protective atmosphere to obtain the deoxynucleoside modified ruthenium complex,
wherein the ruthenium complex azide has a structure represented by Formula II:

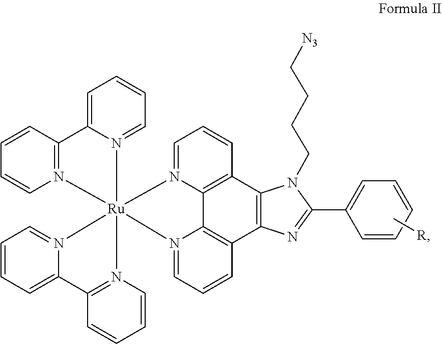

wherein in Formula II, R represents one selected from the group consisting of trifluoromethyl, C₁₋₆ alkyl, alkoxy, halogen, styryl, and nitro-substituted styryl.

In some embodiments, a molar ratio of the ruthenium complex azide to 5-ethynyl-2′-deoxyuridine is in a range of 1:2 to 1:10.

In some embodiments, a molar ratio of the ruthenium complex azide, copper sulfate, and sodium ascorbate is in a range of 100:2:1 to 50:2:1.

In some embodiments, the microwave heating radiation is performed at a temperature of 60° C. to 120° C. for 10 min to 40 min.

In some embodiments, the solvent is a dimethyl sulfoxide aqueous solution.

In some embodiments, the method further comprises, after the microwave heating radiation, subjecting a radiated product obtained after the microwave heating radiation to dilution with water, salting-out, filtration, drying, re-dissolution, purification with a neutral alumina column, and concentration in sequence.

The present disclosure further provides use of the deoxynucleoside modified ruthenium complex described in the above technical solutions in the preparation of a reagent for targeting, recognizing and binding G4 RNA.

The present disclosure further provides use of the deoxynucleoside modified ruthenium complex described in the above technical solutions in the preparation of an antitumor drug.

The present disclosure further provides use of the deoxynucleoside modified ruthenium complex described in the above technical solutions in the preparation of an anti-novel coronavirus drug.

The present disclosure further provides use of the deoxynucleoside modified ruthenium complex described in the above technical solutions in the preparation of a small molecule fluorescent probe.

The present disclosure provides a deoxynucleoside modified ruthenium complex having the ability to target, recognize and bind G4 RNA, especially G4 RNA in novel coronavirus. Therefore, the deoxynucleoside modified ruthenium complex exhibits a potential effect of anti-novel coronavirus, and is expected to developed into an anti-novel coronavirus drug.

The present disclosure further provides a method for preparing the deoxynucleoside modified ruthenium complex described in the above technical solutions, which has advantages of simple operations, fast reaction, low production cost and being environmentally friendly.

### BRIEF DESCRIPTION OF THE DRAWINGS

FIG. 1 shows a mass spectrum of RBL081U.

FIG. 2 shows a mass spectrum of RBL041U.

FIG. 3 shows a mass spectrum of RBL051U.

FIG. 4 shows a mass spectrum of RBL061U.

FIG. 5 shows a mass spectrum of RBL271U.

FIG. 6 shows a mass spectrum of RBL201U.

FIG. 7 shows a mass spectrum of RBL202U.

FIG. 8 shows a mass spectrum of RBL203U.

FIG. 9 shows a mass spectrum of RBLR31U.

FIG. 10 shows a mass spectrum of RBLR31NU.

FIG. 11 shows an ultraviolet (UV) spectrogram of the interaction between RBL201U and ds26.

FIG. 12 shows a UV spectrogram of the interaction between RBL201U and RG-1.

FIG. 13 shows a fluorescence spectrogram of the interaction between RBL201U and ds26.

FIG. 14 shows a fluorescence spectrogram of the interaction between RBL201U and RG-1.

FIG. 15 shows a circular dichroism (CD) spectrogram of the interaction between RBL201U and RG-1.

FIG. 16 shows results of the cellular localization.

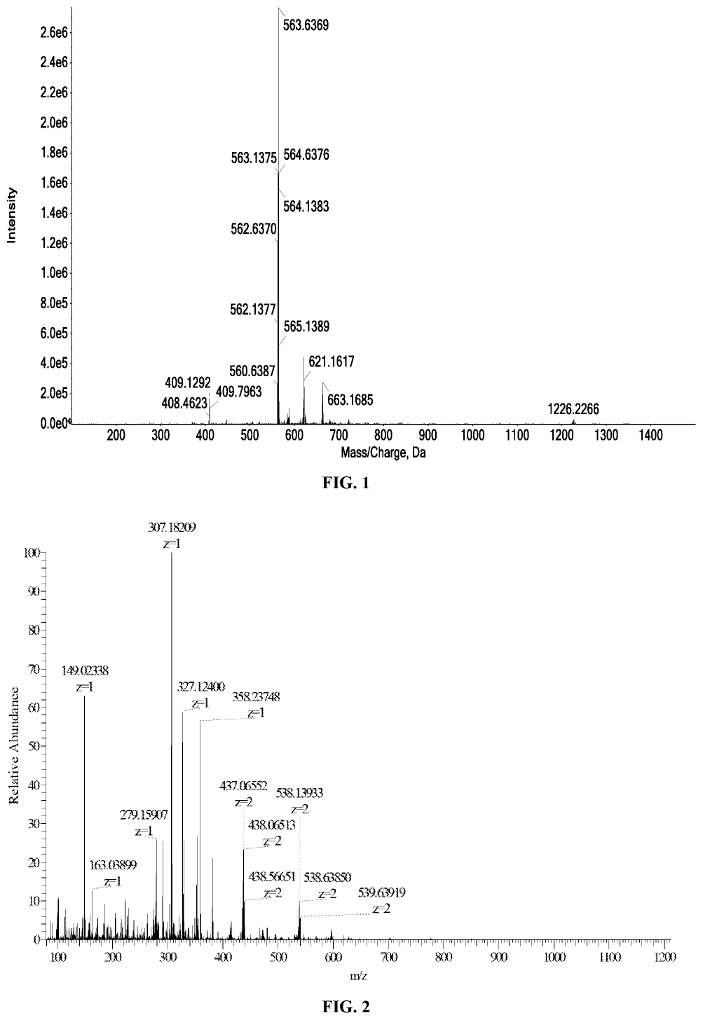

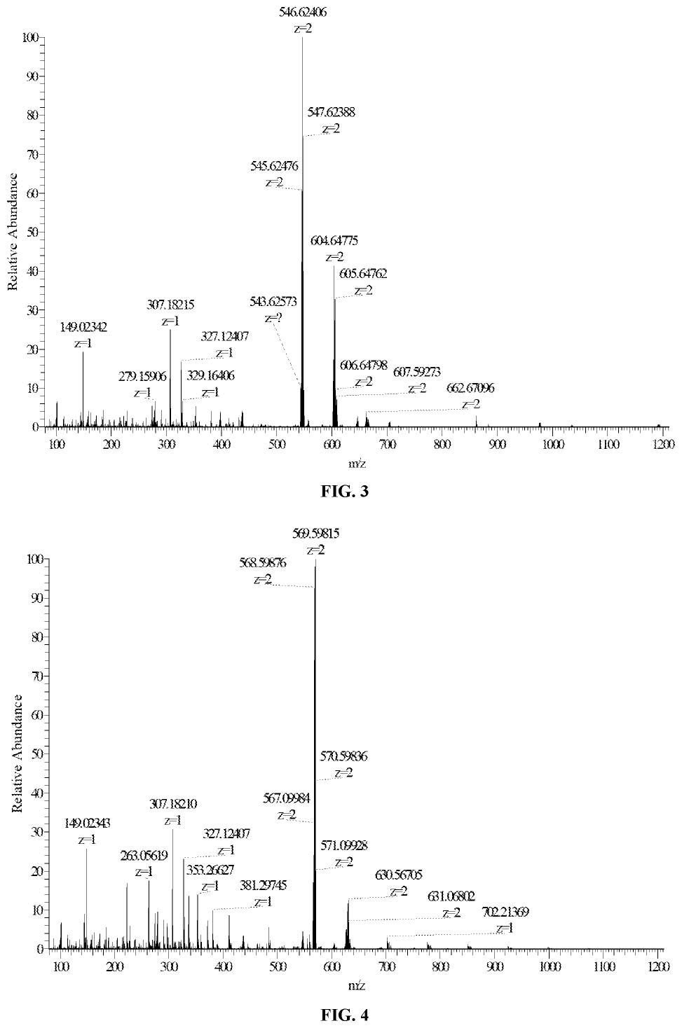

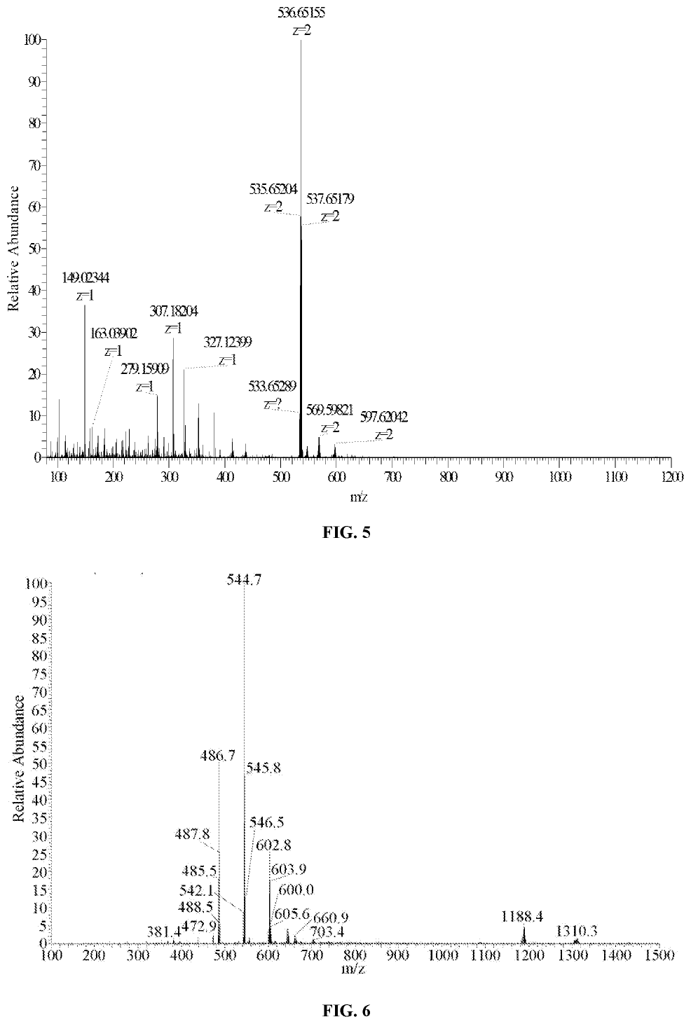

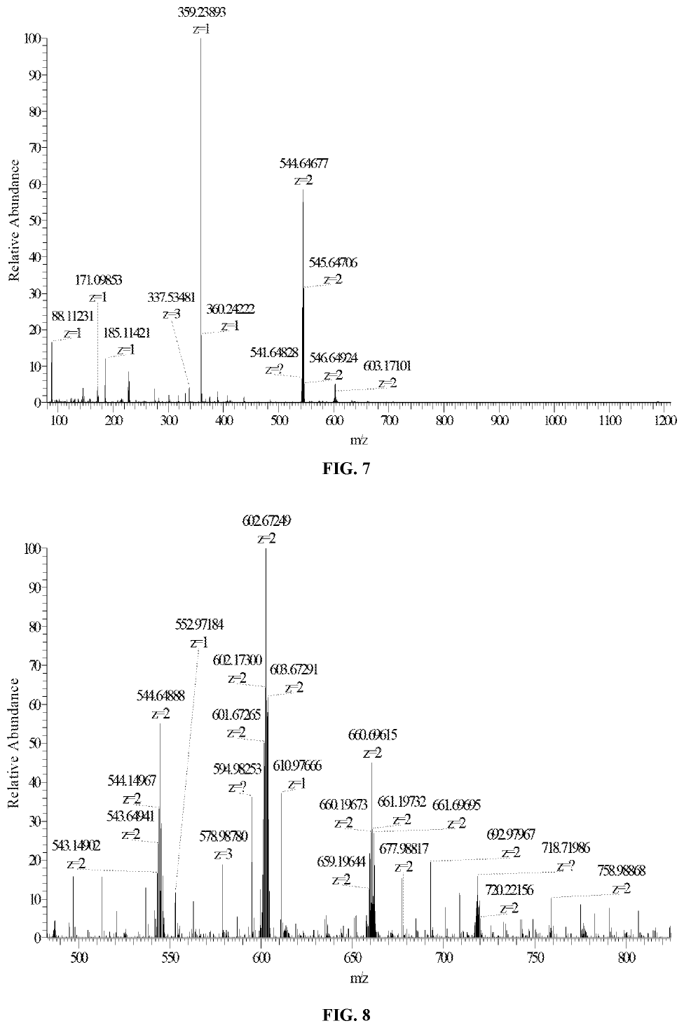

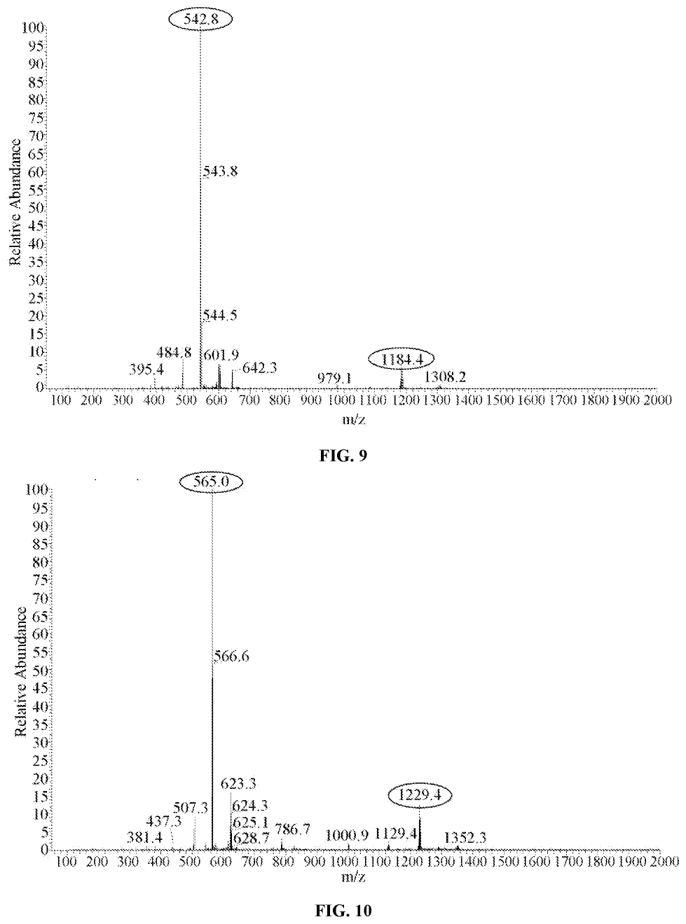

### DETAILED DESCRIPTION OF THE EMBODIMENTS

The present disclosure provides a deoxynucleoside modified ruthenium complex, having a structure represented by Formula I:

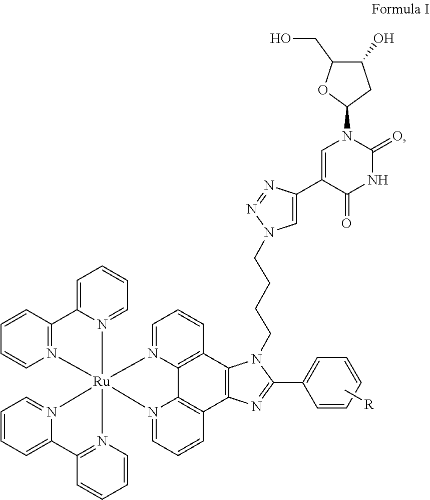

wherein R represents one selected from the group consisting of trifluoromethyl, C₁₋₆ alkyl, alkoxy, halogen, styryl, and nitro-substituted styryl.

In some embodiments of the present disclosure, the deoxynucleoside modified ruthenium complex has a structure represented by any one of Formulas 1 to 10:

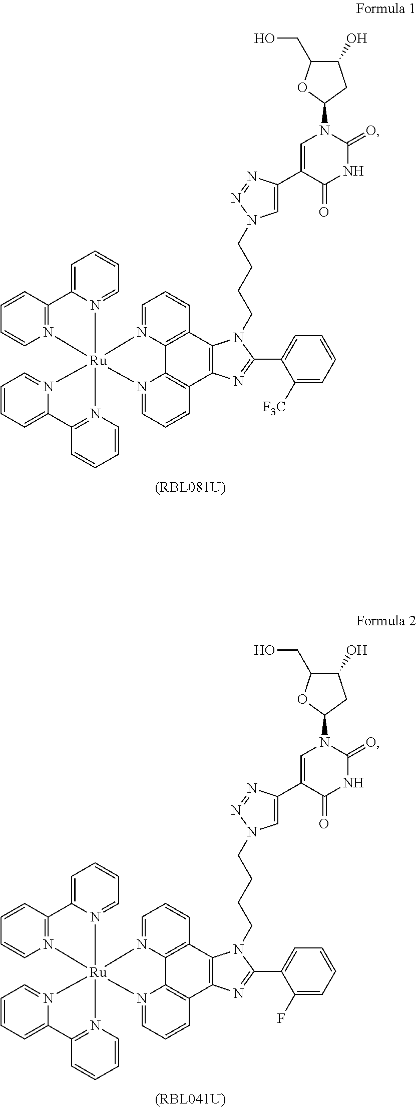

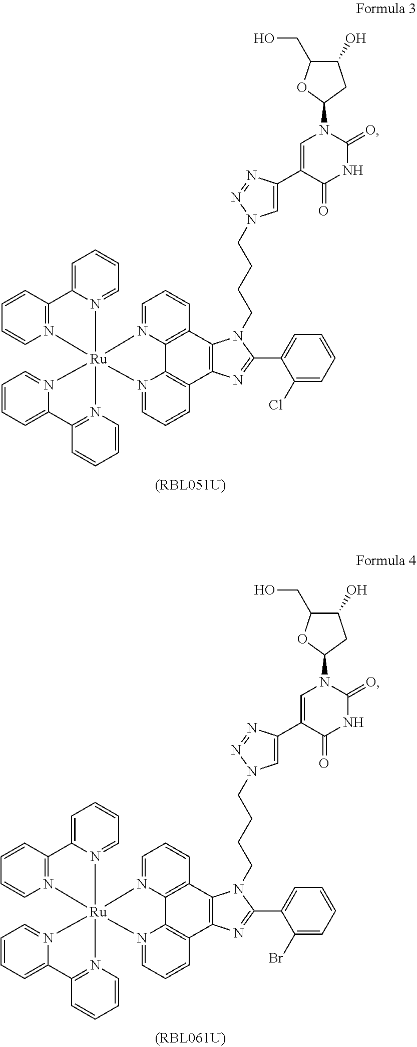

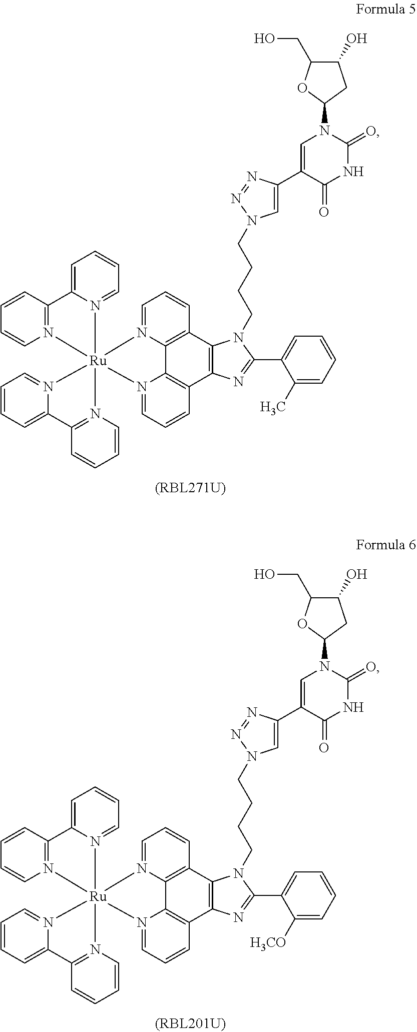

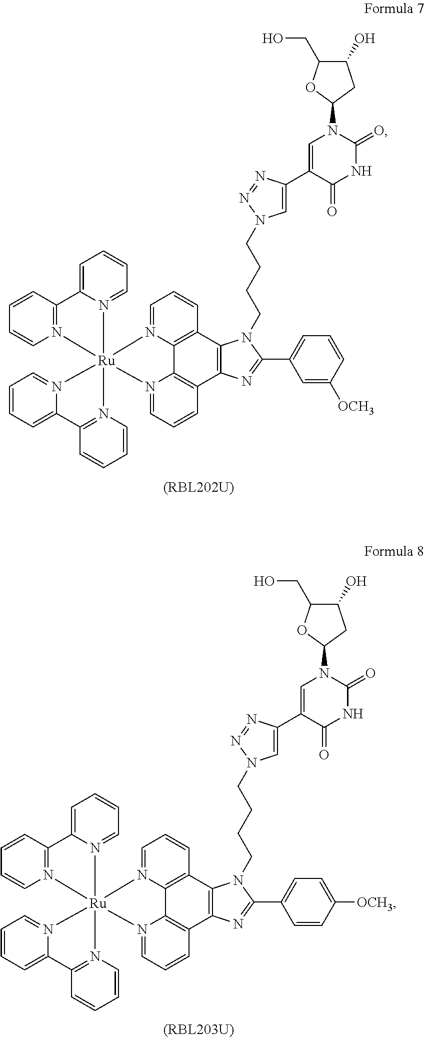

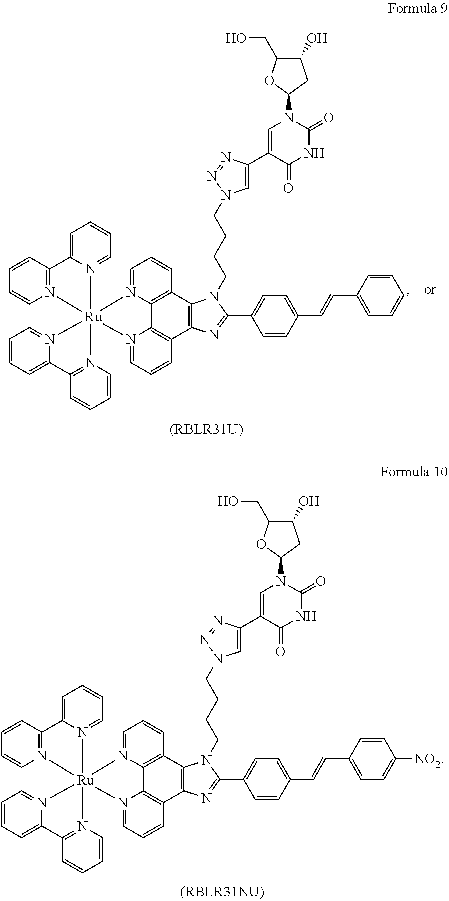

The present disclosure further provides a method for preparing the deoxynucleoside modified ruthenium complex described in the above technical solutions, comprising:
mixing a ruthenium complex azide, 5-ethynyl-2′-deoxyuridine, copper sulfate, sodium ascorbate, and a solvent to obtain a mixture, and subjecting the mixture to microwave heating radiation under a protective atmosphere to obtain the deoxynucleoside modified ruthenium complex,
wherein the ruthenium complex azide has a structure represented by Formula II:

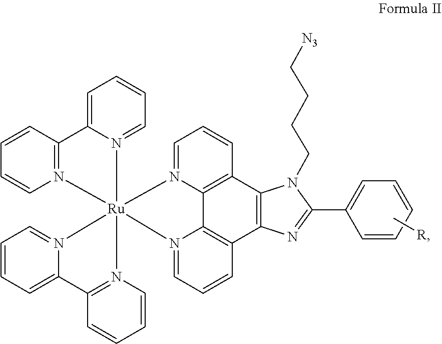

wherein in Formula II, R represents one selected from the group consisting of trifluoromethyl, C₁₋₆ alkyl, alkoxy, halogen, styryl, and nitro-substituted styryl.

In the present disclosure, unless otherwise specified, the raw materials used are commercially available in the art or could be obtained by conventional technical means in the art.

In some embodiments of the present disclosure, the ruthenium complex azide is prepared by a process comprising: mixing an alkane chain modified ruthenium complex, sodium azide, and dimethyl sulfoxide (DMSO) to obtain a mixed solution, subjecting the mixed solution to reaction to obtain the ruthenium complex azide.

In some embodiments of the present disclosure, the alkane chain modified ruthenium complex is the alkane chain modified ruthenium complex prepared according to Chinese patent application No. CN202010620448.9.

There is no special limitation on the amount of DMSO, as long as the raw materials can be mixed evenly.

In some embodiments of the present disclosure, the reaction is performed at ambient temperature, the reaction is performed for 24 h, and the reaction is performed under stirring.

In some embodiments of the present disclosure, after the reaction is completed, the reaction product is filtered to obtain a solid, and the solid is collected as the ruthenium complex azide.

In some embodiments of the present disclosure, a molar ratio of the ruthenium complex azide to 5-ethynyl-2′-deoxyuridine (hereinafter referred as EdU) is in a range of 1:2 to 1:10, and preferably 1:5.

In some embodiments of the present disclosure, a molar ratio of the ruthenium complex azide, copper sulfate, and sodium ascorbate is in a range of 100:2:1 to 50:2:1, and preferably 60:2:1.

In some embodiments of the present disclosure, the solvent is a dimethyl sulfoxide aqueous solution.

In some embodiments of the present disclosure, a mass concentration of dimethyl sulfoxide in the dimethyl sulfoxide aqueous solution is 90%.

In some embodiments of the present disclosure, the protective atmosphere is nitrogen atmosphere, and nitrogen is introduced for 10 min to remove oxygen in the reactor.

In some embodiments of the present disclosure, the microwave heating radiation is performed at a temperature of 60° C. to 120° C., and preferably 90° C. to 100° C. In some embodiments of the present disclosure, the microwave heating radiation is performed for 10 min to 40 min, and preferably 20 min to 30 min.

In some embodiments of the present disclosure, after the microwave heating radiation, the method further comprises subjecting a radiated product obtained after the microwave heating radiation to dilution with water, salting-out, filtration, drying, re-dissolution, purification with a neutral alumina column, and concentration in sequence.

In some embodiments of the present disclosure, the salting-out is performed with sodium perchlorate.

In some embodiments of the present disclosure, the filtration is performed by suction filtration.

In some embodiments of the present disclosure, the drying is performed by vacuum drying.

In some embodiments of the present disclosure, a solvent used for the re-dissolution is acetonitrile.

In some embodiments of the present disclosure, an eluent used for the purification with a neutral alumina column is a mixed solution of acetonitrile and methanol, and a volume ratio of acetonitrile to methanol in the eluent is 5:1.

The present disclosure further provides use of the deoxynucleoside modified ruthenium complex described in the above technical solutions in the preparation of a reagent for targeting, recognizing and binding G4 RNA.

The present disclosure further provides use of the deoxynucleoside modified ruthenium complex described in the above technical solutions in the preparation of an antitumor drug.

The present disclosure further provides use of the deoxynucleoside modified ruthenium complex described in the above technical solutions in the preparation of an anti-novel coronavirus drug.

The present disclosure further provides use of the deoxynucleoside modified ruthenium complex described in the above technical solutions in the preparation of a small molecule fluorescent probe.

In order to further illustrate the present disclosure, the deoxynucleoside modified ruthenium complex and the preparation method and use thereof provided by the present disclosure will be described in detail below with reference to examples, which could not be understood as limiting the scope of protection of the present disclosure.

### Example 1

Preparation of RBL081U:
(1) An alkane chain modified ruthenium complex RBL081-BBr was prepared according to Chinese patent application No. CN202010620448.9:

[Ru(bpy)₂Cl₂]·2H₂O(105 mg, 0.2 mmol), a ligand (0.4 mmol, 2-(2-trifluorophenyl)imidazo[4,5-f][1,10]phenanthroline, CN200910213817.6), and a mixed solvent of ethylene glycol and water (V_{ethylene glycol}:V_{water} 9:1) were added to a three-necked flask, heated to 120° C. in an oil bath and refluxed for 6 h for reaction. After the reaction, the reaction solution was cooled to ambient temperature (25° C.). The resulting system was diluted with water and filtered, obtaining a filtrate. An excess of sodium persulfate was added to the filtrate, obtaining an orange solid in the filtrate. The filtrate was then subjected to suction filtration, obtaining a filter cake. The filter cake was dried, obtaining a first crude product.

The first crude product was dissolved with acetonitrile. The dissolved product was filtered to remove insoluble ligands, obtaining a filtrate. The filtrate was subjected to a neutral alumina column chromatography, in which a mixture of acetonitrile and toluene (V_{acetonitrile}:Vₜₒₗᵤₑₙₑ=2:1) was used as an eluent for elution, obtaining an eluent of the first strip. The eluent of the first strip was collected and dried by rotary evaporation, then re-dissolved in acetonitrile and re-dried by rotary evaporation, obtaining a red solid, i.e., a ruthenium raw material, recorded as compound RBL081.

The compound RBL081 (200 mg, 0.2 mmol) was placed in a 30 mL reaction tube and dissolved with 10 mL of N,N-Dimethylformamide (DMF). 2 g of calcined K₂CO₃ was added to the reaction tube and stirred at ambient temperature for 10 min. 1,4-dibromobutane (4.2 mmol, 500 μL, ρ=1.808 mg/μL, 25° C., M=215 g/mol) was dropped into the reaction tube and stirred at ambient temperature for 5 min, obtaining a mixture. Then the mixture was subjected to reaction under the condition of an oil bath heating at 90° C. for 6 h. After the reaction, the reaction solution was filtered through a glass funnel, obtaining a first filter cake and a filtrate. The first filter cake was washed with acetonitrile until the percolate was free of red color to achieve the reuse of potassium carbonate. The filtrate was added with a 5-times volume amount of water and extracted with methyl tert-butyl ether three times to remove excess 1,4-dibromobutane, obtaining a bottom layer liquid (aqueous layer). After the extraction, sodium perchlorate was added to the bottom layer liquid to precipitate a crystal. The resulting solution was subjected to suction filtration with a Buchner funnel, obtaining a second filter cake. The second filter cake was collected as a second crude product.

The second crude product was dissolved with acetonitrile, and then subjected to neutral alumina column chromatography, in which a mixture of acetonitrile and toluene (V_{acetonitrile}:Vₜₒₗᵤₑₙₑ=2:1) was used as an eluent for elution, obtaining an eluent of the first strip. The eluent of the first strip was collected and dried by rotary evaporation, and then re-dissolved in acetonitrile and re-dried by rotary evaporation, obtaining a red solid, i.e., RBL081-BBr.

(2) Preparation of RBL081-N₃

RBL081-BBr (0.7 mmol) and sodium azide (1.4 mmol) were weighed and added into a 50 mL reactor, and 50 mL of DMSO was added to the reactor for dissolution. The resulting mixed solution was stirred at ambient temperature for 24 h, obtaining a mixture.

After the reaction, an equal volume of water and sodium perchlorate (1 mmol) were added to the mixture. The resulting mixture was filtered, obtaining a solid. The solid was collected as a RBL081-N₃.

(3) Preparation of RBL081U

RBL081-N₃ (0.2 mmol) and EdU (1 mmol) were weighed and added to a 30 mL reaction tube. Copper sulfate (0.06 mmol), sodium ascorbate (0.12 mmol), and 10 mL of a 90% DMSO aqueous solution were added to the reaction tube for dissolution, and mixed to be uniform, obtaining a mixture.

After introducing N₂ for 10 min, the mixture was subjected to microwave radiation at 90° C. for 30 min, obtaining a reaction system.

After the reaction, the reaction system was diluted with 50 mL of deionized water, subjected to precipitation with sodium perchlorate (1 mmol), standing, and then suction filtration, obtaining a solid. The solid was collected and dried in a vacuum, obtaining a third crude product.

After being dried, the third crude product was dissolved with acetonitrile, and purified on a neutral alumina column, in which a mixture of acetonitrile and methanol (a volume ratio of acetonitrile and methanol being 5:1) was used for elution, obtaining an eluent of a target strip. The eluent of the target band was collected and concentrated, obtaining a red solid, i.e., the target product with a structure represented by Formula 1, recorded as RBL081U. ESI-MS: Cal. 563.5, Found. 563.6369 (½[M-2ClO4⁻]).

### Example 2

Preparation of RBL041U:

The preparation method was the same as Example 1, except that 2-(2-trifluorophenyl)imidazo[4,5-f][1,10]phenanthroline in Example 1 was replaced with 2-(2-fluorophenyl)imidazo[4,5-f][1,10]phenanthroline, obtaining RBL041, RBL081 was replaced with RBL041, obtaining RBL041-BBr, RBL081-BBr was replaced with RBL041-BBr, obtaining RBL041-N₃, and RBL081-N₃ was replaced with RBL041-N₃, obtaining a target product represented by Formula 2, recorded as RBL041U. ESI-MS: Cal. 538.5, Found. 538.13933 (½[M-2ClO4⁻]).

### Example 3

Preparation of RBL051U:

The preparation method was the same as Example 1, except that 2-(2-trifluorophenyl)imidazo[4,5-f][1,10]phenanthroline in Example 1 was replaced with 2-(2-chlorophenyl)imidazo[4,5-f][1,10]phenanthroline, obtaining RBL051, RBL081 was replaced with RBL051, obtaining RBL051-BBr, RBL081-BBr was replaced with RBL051-BBr, obtaining RBL051-N₃, and RBL081-N₃ was replaced with RBL051-N₃, obtaining a target product represented by Formula 3, recorded as RBL051U. ESI-MS: Cal. 546.5, Found. 546.62406 (½[M-2ClO4^{-]).}

### Example 4

Preparation of RBL061U:

The preparation method was the same as Example 1, except that 2-(2-trifluorophenyl)imidazo[4,5-f][1,10]phenanthroline in Example 1 was replaced with 2-(2-bromophenyl)imidazo[4,5-f][1,10]phenanthroline, obtaining RBL061, RBL081 was replaced with RBL061, obtaining RBL061-BBr, RBL081-BBr was replaced with RBL061-BBr, obtaining RBL061-N₃, and RBL081-N₃ was replaced with RBL061-N₃, obtaining a target product represented by Formula 4, recorded as RBL061U. ESI-MS: Cal. 568.5, Found. 568.59876 (½[M-2ClO4^{-]).}

### Example 5

Preparation of RBL271U:

The preparation method was the same as Example 1, except that 2-(2-trifluorophenyl)imidazo[4,5-f][1,10]phenanthroline in Example 1 was replaced with 2-(2-methylphenyl)imidazo[4,5-f][1,10]phenanthroline, obtaining RBL271, RBL081 was replaced with RBL271, obtaining RBL271-BBr, RBL081-BBr was replaced with RBL271-BBr, obtaining RBL271-N₃, and RBL081-N₃ was replaced with RBL271-N₃, obtaining a target product represented by Formula 5, recorded as RBL271U. ESI-MS: Cal. 536.5, Found. 536.65155 (½[M-2ClO4^{-]).}

### Example 6

Preparation of RBL201U:

The preparation method was referred to as Example 1, excepting that 2-(2-trifluorophenyl)imidazo[4,5-f][1,10]phenanthroline in Example 1 was replaced with 2-(2-methoxyphenyl)imidazo[4,5-f][1,10]phenanthroline, obtaining RBL201, RBL081 was replaced with RBL201, obtaining RBL201-BBr, RBL081-BBr was replaced with RBL201-BBr, obtaining RBL201-N₃, and RBL081-N₃ was replaced with RBL201-N₃, obtaining a target product represented by Formula 6, recorded as RBL201U. ESI-MS: Cal. 544.5, Found. 544.5 (½[M-2ClO4⁻]).

### Example 7

Preparation of RBL202U:

The preparation method was the same as Example 1, except that 2-(2-trifluorophenyl)imidazo[4,5-f][1,10]phenanthroline in Example 1 was replaced with 2-(3-methoxyphenyl)imidazo[4,5-f][1,10]phenanthroline, obtaining RBL202, RBL081 was replaced with RBL202, obtaining RBL202-BBr, RBL081-BBr was replaced with RBL202-BBr, obtaining RBL202-N₃, and RBL081-N₃ was replaced with RBL202-N₃, obtaining a target product represented by Formula 7, recorded as RBL202U. ESI-MS: Cal. 544.5, Found. 544.64677 (½[M-2ClO4⁻]).

### Example 8

Preparation of RBL203U:

The preparation method was the same as Example 1, except that 2-(2-trifluorophenyl)imidazo[4,5-f][1,10]phenanthroline in Example 1 was replaced with 2-(4-methoxyphenyl)imidazo[4,5-f][1,10]phenanthroline, obtaining RBL203, RBL081 was replaced with RBL203, obtaining RBL203-BBr, RBL081-BBr was replaced with RBL203-BBr, obtaining RBL203-N₃, and RBL081-N₃ was replaced with RBL203-N₃, obtaining a target product represented by Formula 8, recorded as RBL203U. ESI-MS: Cal. 544.5, Found. 544.64888 (½[M-2ClO4⁻]).

### Example 9

Preparation of RBLR31U:

The preparation method was the same as Example 1, except that 2-(2-trifluorophenyl)imidazo[4,5-f][1,10]phenanthroline in Example 1 was replaced with 2-styryl-imidazo[4,5-f][1,10]phenanthroline, obtaining RBLR31, RBL081 was replaced with RBLR31, obtaining RBLR31-BBr, RBL081-BBr was replaced with RBLR31-BBr, obtaining RBLR31-N₃, and RBL081-N₃ was replaced with RBLR31-N₃, obtaining a target product represented by Formula 9, recorded as RBLR31U. ESI-MS: Cal. 542.5, Found. 542.8 (½[M-3ClO4⁻+Na⁺]). Cal. 1184, Found. 1184.4 ([M-2ClO4⁻+Na⁺]).

### Example 10

Preparation of RBLR31NU:

The preparation method was the same as Example 1, except that 2-(2-trifluorophenyl)imidazo[4,5-f][1,10]phenanthroline in Example 1 was replaced with 2-(4-nitrostyryl)-imidazo[4,5-f][1,10]phenanthroline, obtaining RBLR31N₃, RBL081 was replaced with RBLR31N₃ obtaining RBLR31N-BBr, RBL081-BBr was replaced with RBLR31N-BBr, obtaining RBLR31N-N₃, and RBL081-N₃ was replaced with RBLR31N-N₃, obtaining a target product represented by Formula 10, recorded as RBLR31NU. ESI-MS: Cal. 565, Found. 565.0 (½[M-3ClO4⁻+Na⁺]). Cal. 1129, Found. 1129.4 ([M-2ClO4⁻+Na⁺]).

### Example 11

Toxicity Test of Compounds on Cells:
1. Seeding plates: Cells were treated and resuspended in culture mediums after passaging, obtaining a cell suspension. 10 μL of a cell suspension was taken and the number of cells therein was counted as n. Each 96-well plate needed 1×10⁵/n of the cell suspension. The cell suspension and 10 mL of a culture medium were added to a loading slot and mixed to be uniform. A capacity of a multi-channel pipette gun was adjusted to 100 μL to seed a plate. The 96-well plates were placed in a CO₂ incubator for 24 h for incubation.
2. Adding a drug: 1.5 mL EP tubes were used. The culture medium was used to prepare a 700 μL sample with a concentration of 2 times the highest concentration. Samples with gradient concentrations were prepared by diluting the culture medium in half. The prepared samples were added to the 96-well plate sequentially from low concentration to high concentration, and the dosage is 100 μL per well. The last row of the 96-well plate was set as a blank control group. The 96-well plate was placed in a CO₂ incubator for 72 h for incubation.
3. Adding MTT: The 96-well plate was taken out. 20 μL of an MTT solution (5 mg/mL) was added to each well. The 96-well plate was placed in a CO₂ incubator for 4 h for incubation.
4. Collecting the plate: The 96-well plate was taken out. The liquid in the wells was sucked out with a vacuum pump. 150 μL of a DMSO solution was added to each well. The absorbance value of a solution was tested at 570 nm excitation wavelength by an enzyme calibration.

The cells used in the laboratory were Bears-2B, QSG-7701, HepG2, and A549, and the experimental results are shown in Table 1. It can be seen from the experimental results that this series of drugs have no obvious toxicity to normal human hepatocytes and pulmonary cells, and have desired anti-tumor activity and selectivity for liver cancer and lung cancer, and desired drug-forming properties, indicating an expectation to be developed into an anti-tumor drug.

TABLE 1
Toxicity test results of the deoxynucleoside
modified ruthenium complexes on cells

| | Bears-2B | QSG-7701 | HepG2 | A549 |
|---|---|---|---|---|
| RBL081U | >100 | >100 | 88.71 ± 3.51 | 2.34 ± 0.02 |
| RBL041U | >100 | >100 | 50.79 ± 0.85 | 2.31 ± 0.03 |
| RBL051U | >100 | >100 | 25.84 ± 0.41 | 2.95 ± 0.04 |
| RBL061U | >100 | >100 | 14.60 ± 0.42 | 5.04 ± 0.71 |
| RBL271U | >100 | >100 | 25.20 ± 0.40 | 5.62 ± 0.08 |
| RBL201U | >100 | >100 | 6.73 ± 0.09 | 6.49 ± 0.14 |
| RBL202U | >100 | >100 | 26.11 ± 0.37 | 10.71 ± 0.06 |
| RBL203U | >100 | >100 | 29.85 ± 0.72 | 10.84 ± 0.28 |
| RBLR31U | >100 | >100 | 19.73 ± 0.42 | 12.38 ± 0.13 |
| RBLR31NU | >100 | >100 | 12.99 ± 0.23 | 12.99 ± 0.23 |

### Example 12

Interaction between the compound and G4 RNA:

RBL201U was selected for subsequent experiments of G4 RNA. G4 RNA was selected as RG-1, a G4 RNA in the sequence of novel coronavirus based on the literature “Targeting RNA G-Quadruplex in SARS-CoV-2: A Promising Therapeutic Target for COVID-19?”. All the complexes mentioned in the subsequent experiments were RBL201U, and all the G4 RNAs were RG-1.

UV Titration

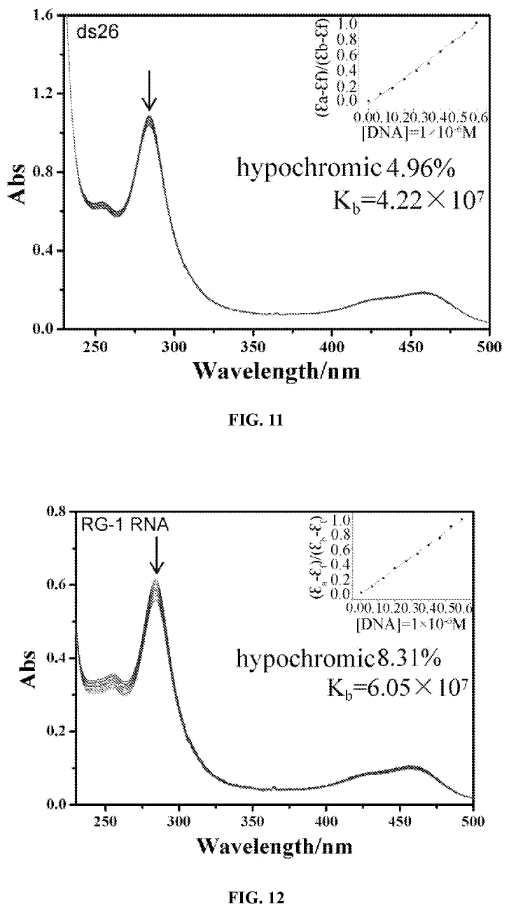

3 mL of a buffer solution was added to a control pool. 3 mL of 20 μM complex solution prepared with a buffer solution was added to a reference pool. A G4 RNA and ds26 (double-stranded DNA) solution with the same volume were respectively added to the two pools every 3 min with a micro-sampler (after each addition, the resulting solution needed to be mixed with a capillary or a pipette gun), such that the concentration ratios of RNA/DNA to the complex were increased at a certain ratio. The changes in the electronic absorption spectra of the complex in the range of 200 nm to 800 nm were detected, as shown in FIGS. 11-12. FIG. 11 shows a UV spectrogram of the interaction between RBL201U and ds26. FIG. 12 shows a UV spectrogram of the interaction between RBL201U and RG-1. It can be seen from the experimental results that the binding constant Kb of the complex with RG-1 is 6.05×10⁷, and the binding constant Kb of the complex with ds26 is 4.22×10⁷. Moreover, the complex exhibits a significant hypochromic effect after being bound to G4 RNA (RG-1). Compared with the double-stranded DNA, the complex exhibits a more strongly binding to G4 RNA.

Fluorescence Titration

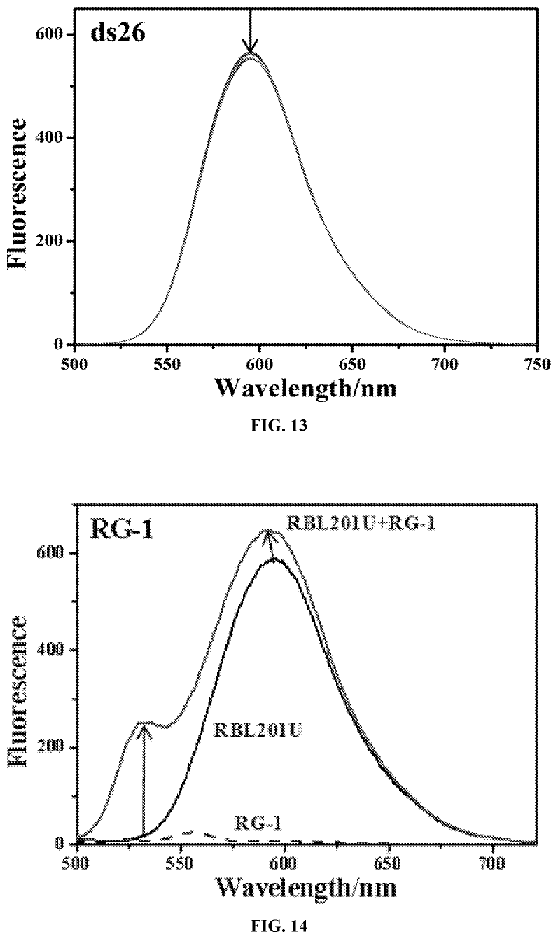

5 mL of 10 μM complex solution was prepared. 3 mL of the complex solution was added to a fluorescence cuvette for fluorescence scanning. 2 μL of RNA or DNA solution was added to the fluorescence cuvette every 3 min with a micro-sampler and mixed to be uniform. Then the changes in fluorescence emission peaks with the addition of RNA or DNA were measured, as shown in FIGS. 13-14. FIG. 13 shows a fluorescence spectrogram of the interaction between RBL201U and ds26. FIG. 14 shows a fluorescence spectrogram of the interaction between RBL201U and RG-1. It can be seen from the experimental results that the fluorescence intensity of the complex bound to double-stranded DNA (ds26) is almost unchanged. However, a new emission peak appears at 525 nm after the complex bound to G4 RNA (RG-1), and the intensity thereof is enhanced with the increase of RNA, indicating that the hydrophobicity is enhanced after the complex bound to G4 RNA and a hydrophobic structure may be formed, and also indicating that compared with the double-stranded DNA, the complex obviously selectively binds to G4 RNA.

CD Titration

3 mL of a buffer solution was added to a cuvette. Under the condition that the transparent side of the cuvette faced the direction of the light source, the cuvette was placed into an instrument to read a spectrogram for deducting background.

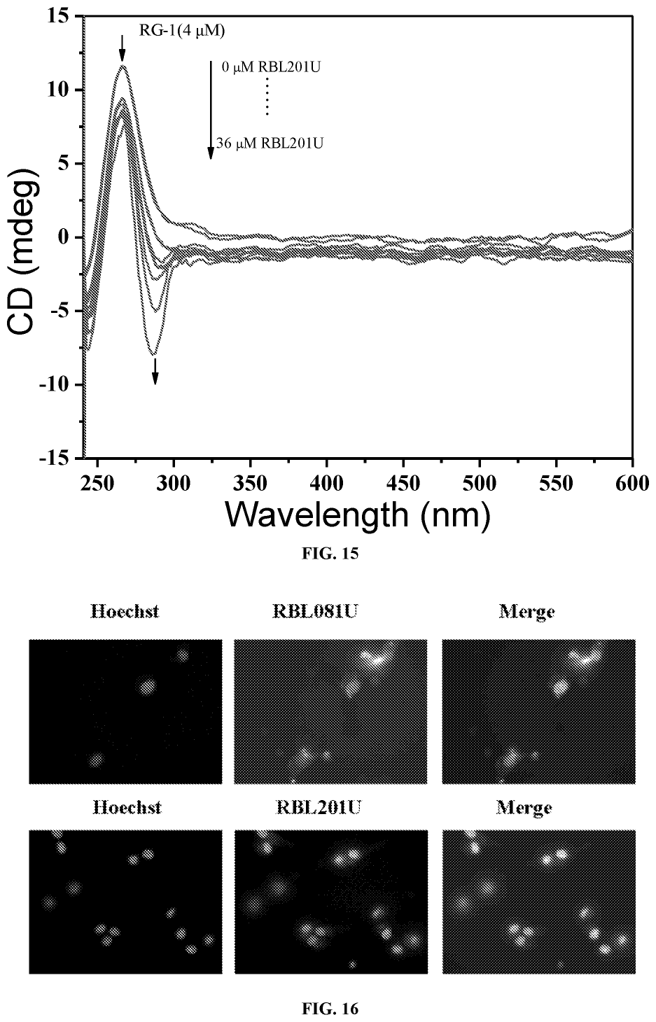

60 μL of G4 RNA (100 μM) was diluted to a 2 μM drug solution. The drug solution was mixed to be uniform and tested to obtain a spectrogram. Complex solutions with the same volume were added to the cuvette every 3 min with a micro-sampler, and each addition needed to be mixed with a capillary or a pipette gun. The concentrations of the complex and G4 RNA were increased at a certain ratio. The changes of the CD spectrum were observed in the wavelength range until the spectrum was unchanged. FIG. 15 shows a CD spectrogram of the interaction between RBL201U and RG-1. It can be seen from the experimental results that with the increase in the concentration ratio of the complex to G4 RNA, a new negative signal peak appears at 285 nm, indicating that the binding mode of the complex to G4 RNA is mainly insertion binding.

It can be seen from the above experiment results that the complex RBL201U may selectively bind to G4 RNA (RG-1), and the binding mode of the them is insertion binding. The binding of the complex with RG-1 may be able to block process such as viral translation, thus exhibiting the effect of anti-coronavirus.

### Example 13 Cell Localization

HepG2 cell species that grew well in the logarithmic phase were taken into confocal capsules, where each capsule contained about 5×10⁴ cells. The cells in the confocal capsules were incubated in an incubator for 24 h for cell adherence. After incubating for 24 h, drug mother liquors were diluted to the corresponding concentrations. The adhered cells were taken out and treated by adding drugs according to the corresponding concentrations. The capsules were covered with lids, cross-shaken to even, then marked and placed in the incubator for 72 h for continuous incubation. A staining solution was prepared by mixing hoechst 33258 nucleic acid dye with DMEM culture medium in a ratio of 1:1000. 500 μL of the staining solution was added to each capsule. The cells in the capsules were incubated and dyed in the incubator for 30 min. After the incubation, the staining solution was suctioned and discarded. The capsules were added with 1 mL of phosphate buffer saline (PBS), observed, and took pictures under a fluorescence microscope.

As shown in FIG. 16, the red fluorescence of the complex basically overlapped with the blue fluorescence of the nucleus dye, indicating that this type of complex may be well localized to the nucleus and have an expectation to be a fluorescent molecular probe for localizing the nucleus.

The above descriptions are only preferred embodiments of the present disclosure but not limitations in any form. It should be noted by those skilled in the art that without deviating from the principle of the present disclosure, various improvements or modifications can be made, and these improvements or modifications should also be regarded as the scope of the present disclosure.

1. A deoxynucleoside modified ruthenium complex, having a structure represented by Formula I: 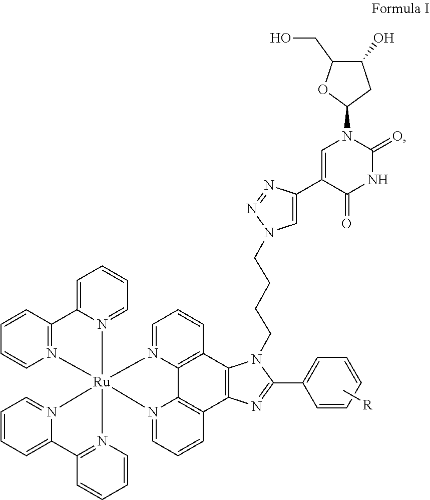
wherein R represents one selected from the group consisting of trifluoromethyl, C₁₋₆ alkyl, alkoxy, halogen, styryl, and nitro-substituted styryl.

2. The deoxynucleoside modified ruthenium complex of claim 1, having a structure represented by any one of Formulas 1 to 10:

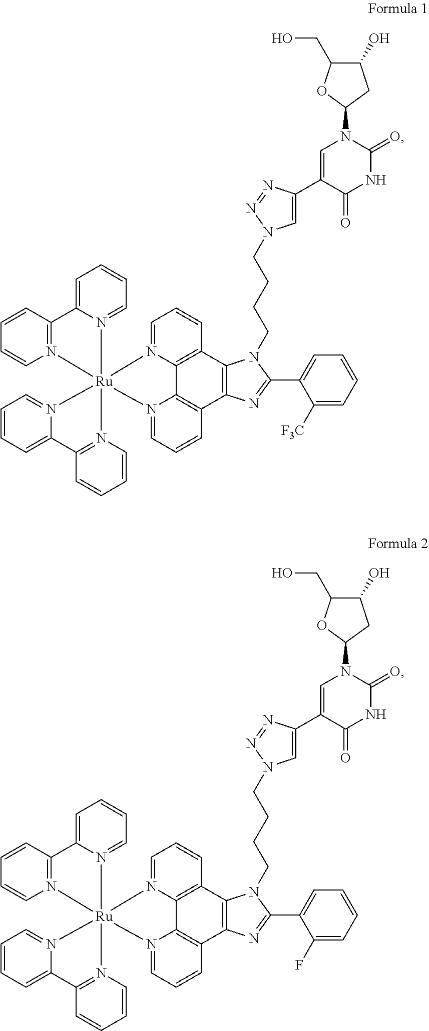

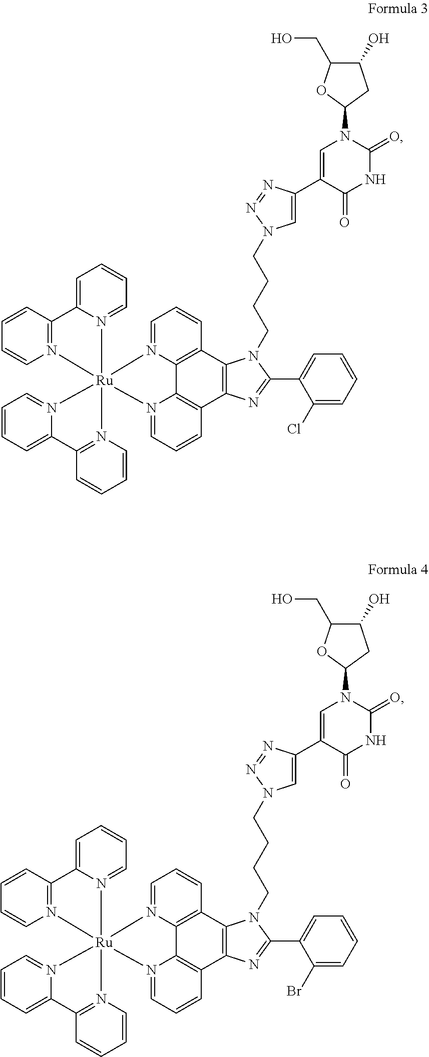

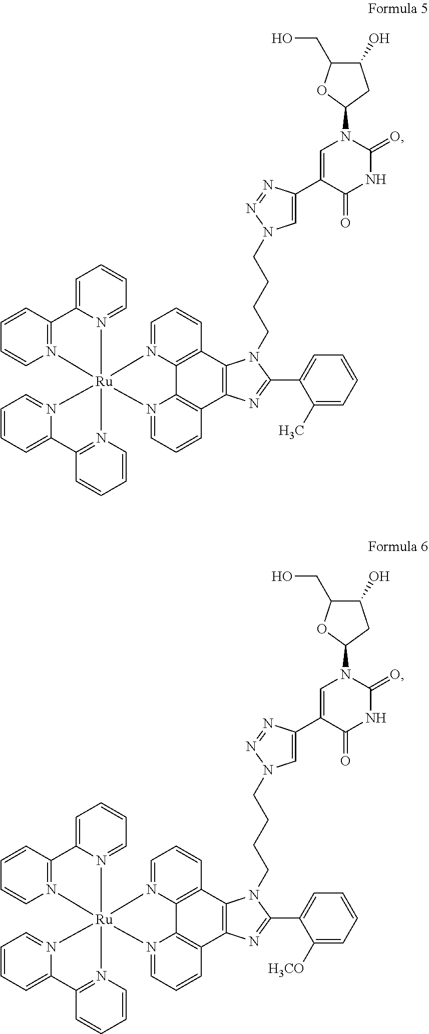

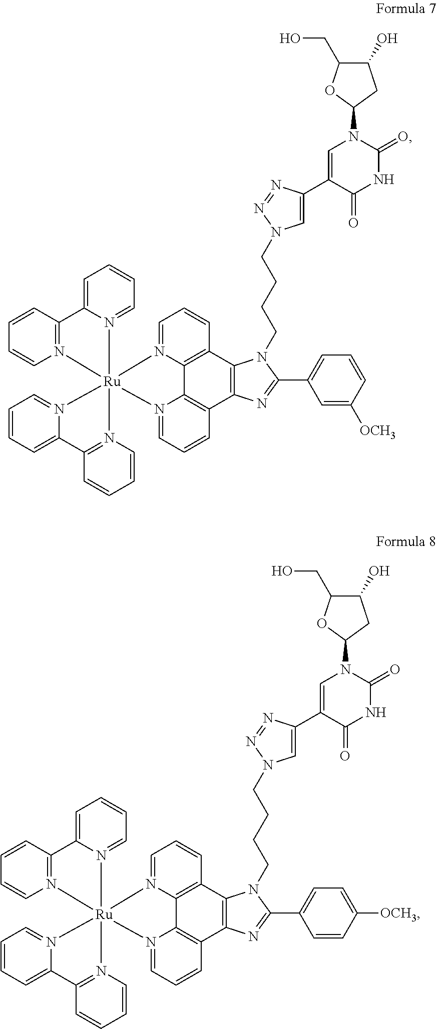

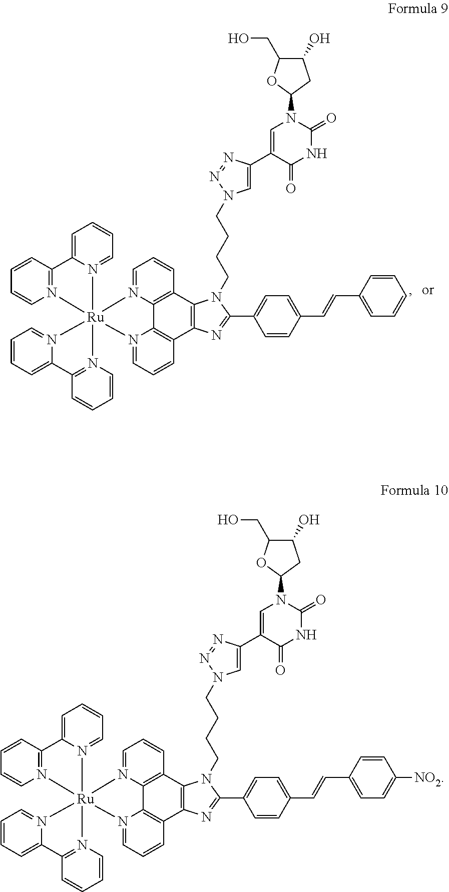

3. A method for preparing the deoxynucleoside modified ruthenium complex of claim 1, comprising:
mixing a ruthenium complex azide, 5-ethynyl-2′-deoxyuridine, copper sulfate, sodium ascorbate, and a solvent to obtain a mixture, and subjecting the mixture to microwave heating radiation under a protective atmosphere to obtain the deoxynucleoside modified ruthenium complex,
wherein the ruthenium complex azide has a structure represented by Formula II: 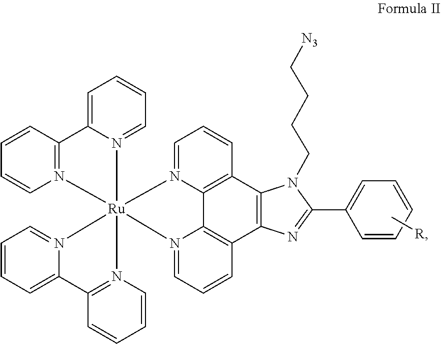
wherein in Formula II, R represents one selected from the group consisting of trifluoromethyl, C1-6 alkyl, alkoxy, halogen, styryl, and nitro-substituted styryl.

4. The method of claim 3, wherein a molar ratio of the ruthenium complex azide to 5-ethynyl-2′-deoxyuridine is in a range of 1:2 to 1:10.

5. The method of claim 3, wherein the microwave heating radiation is performed at a temperature of 60° C. to 120° C. for 10 min to 40 min.

6. The method of claim 3, wherein the method further comprises, after the microwave heating radiation, subjecting a radiated product obtained after the microwave heating radiation to dilution with water, salting-out, filtration, drying, re-dissolution, purification with a neutral alumina column, and concentration in sequence.

7. A method for targeting, recognizing and binding G4 RNA, comprising using the deoxynucleoside modified complex of claim 1.

8. A method for preparing an antitumor drug, comprising using the deoxynucleoside modified ruthenium complex of claim 1.

9. A method for preparing an anti-novel coronavirus drug, comprising using the deoxynucleoside modified ruthenium complex of claim 1.

10. A method for preparing a small molecule fluorescent probe, comprising using the deoxynucleoside modified ruthenium complex of claim 1.

11. The method of claim 3, wherein the deoxynucleoside modified ruthenium complex has a structure represented by any one of Formulas 1 to 10:

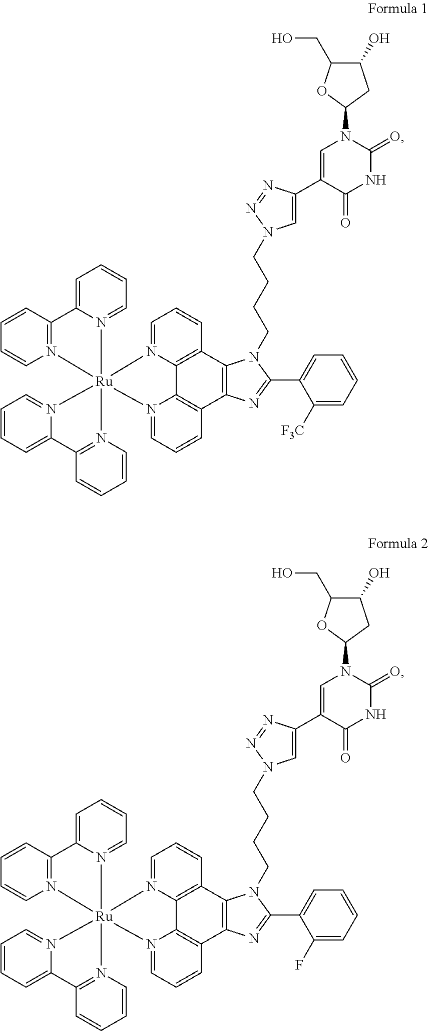

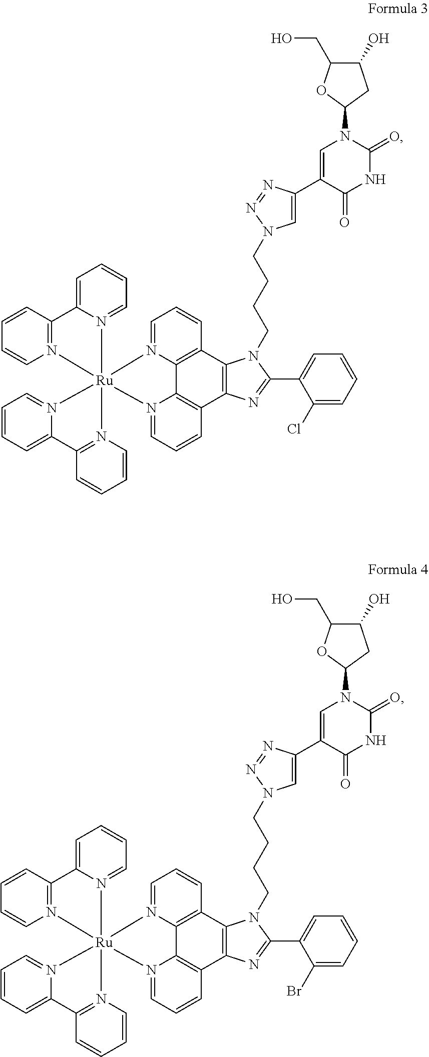

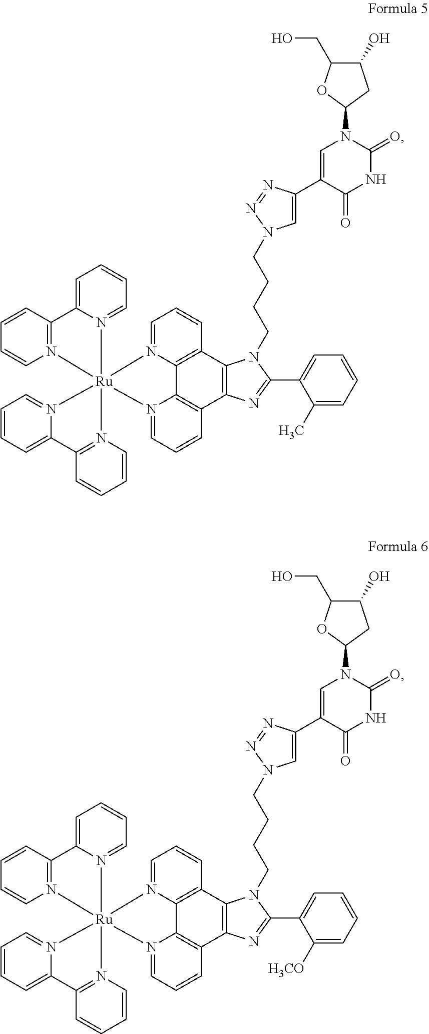

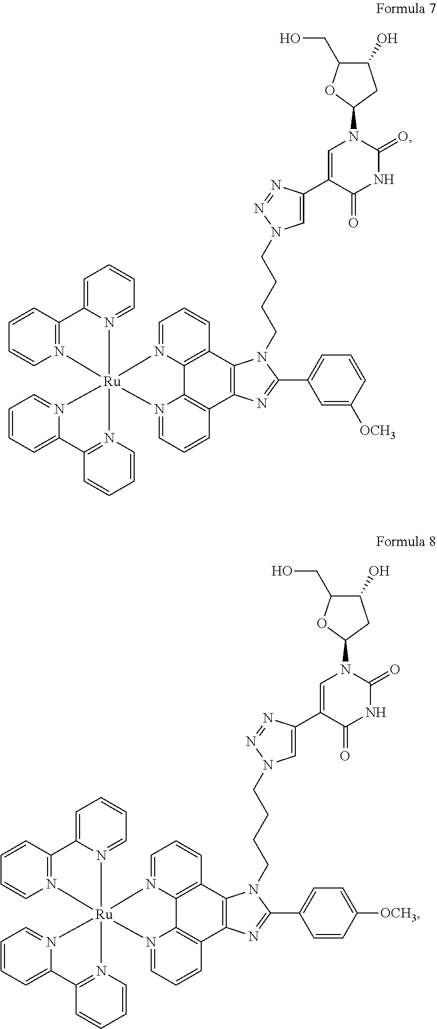

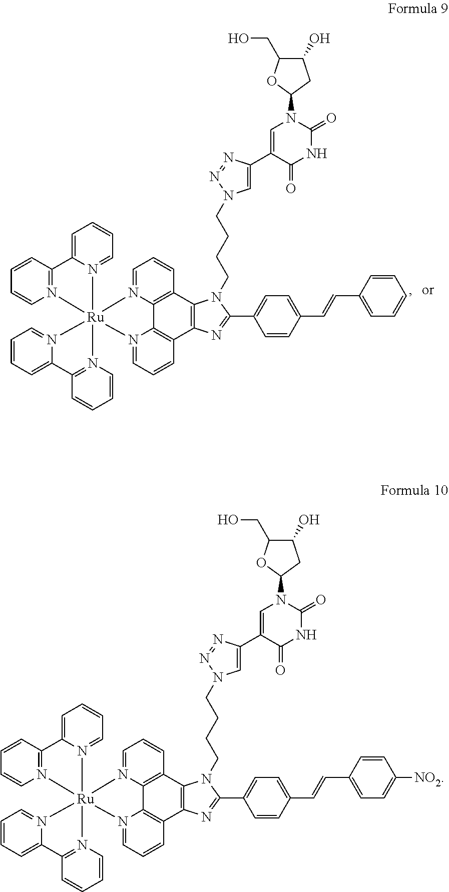

12. The method of claim 7, wherein the deoxynucleoside modified ruthenium complex has a structure represented by any one of Formulas 1 to 10:

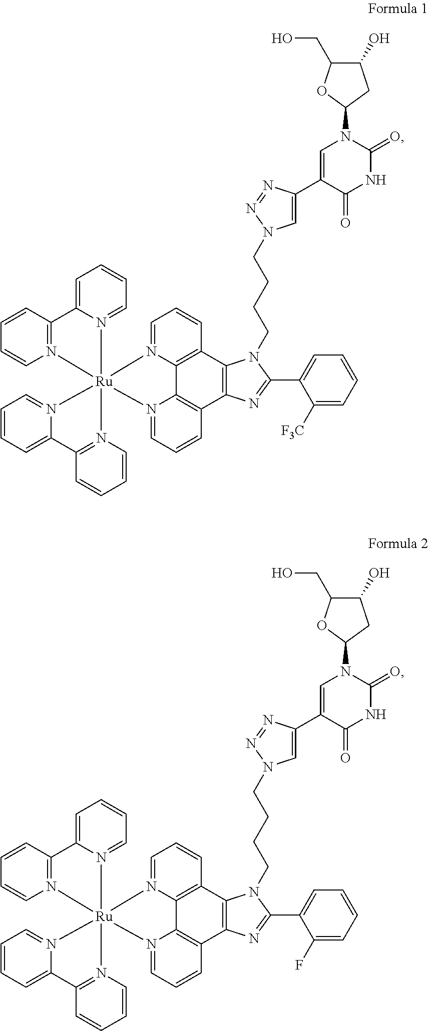

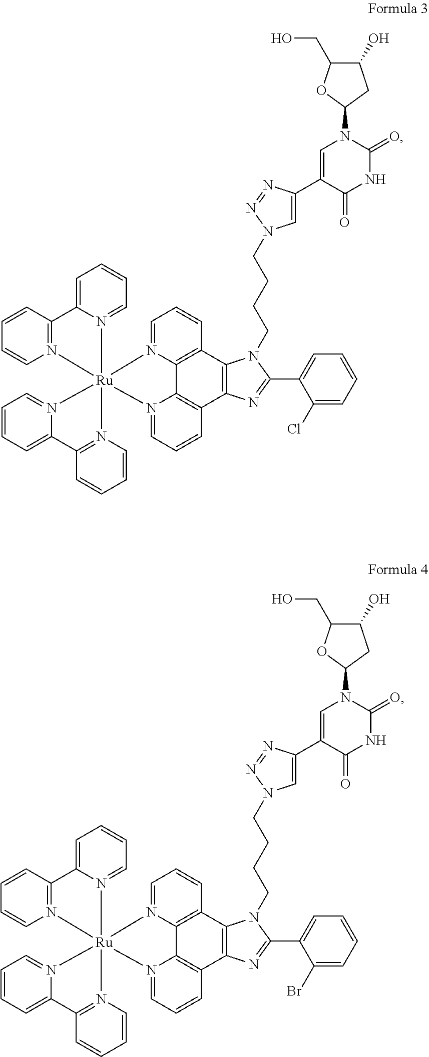

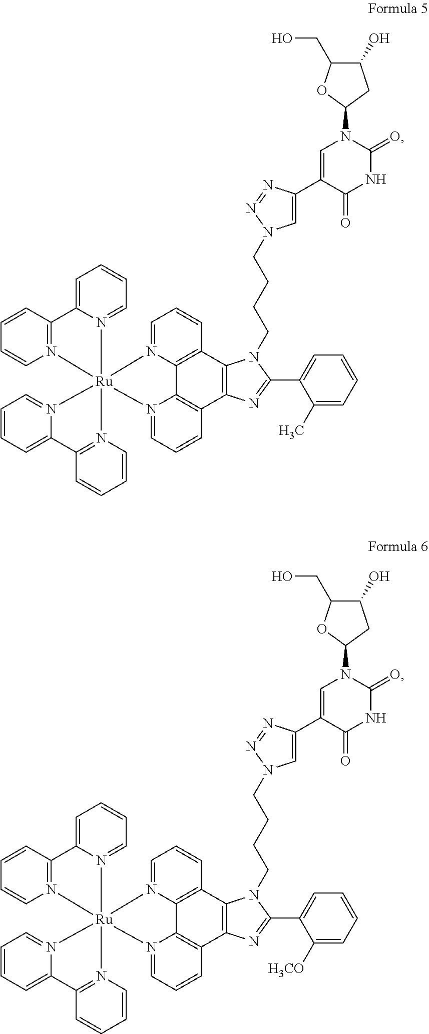

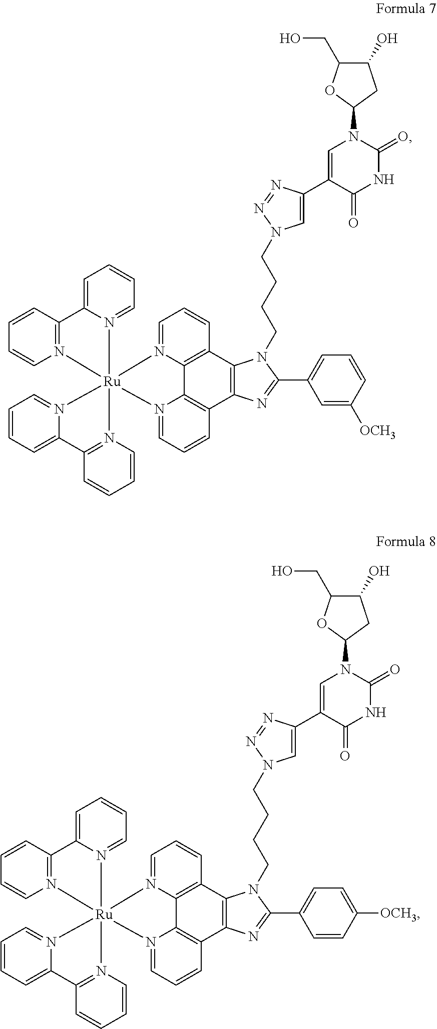

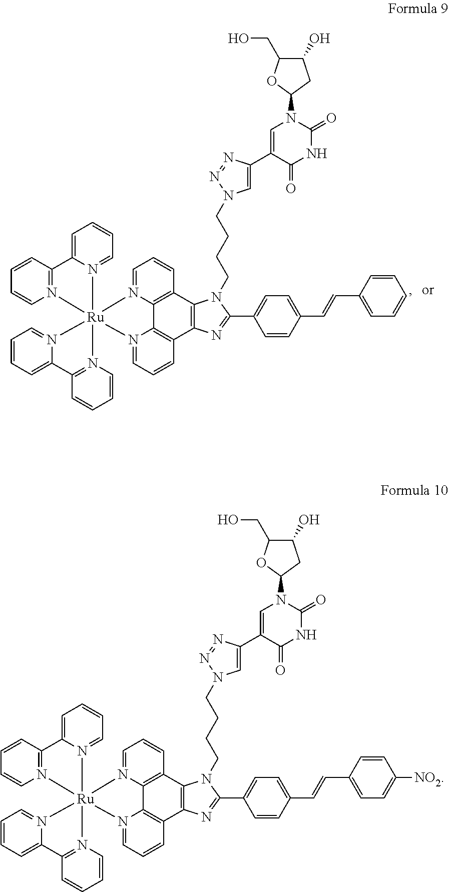

13. The method of claim 8, wherein the deoxynucleoside modified ruthenium complex has a structure represented by any one of Formulas 1 to 10:

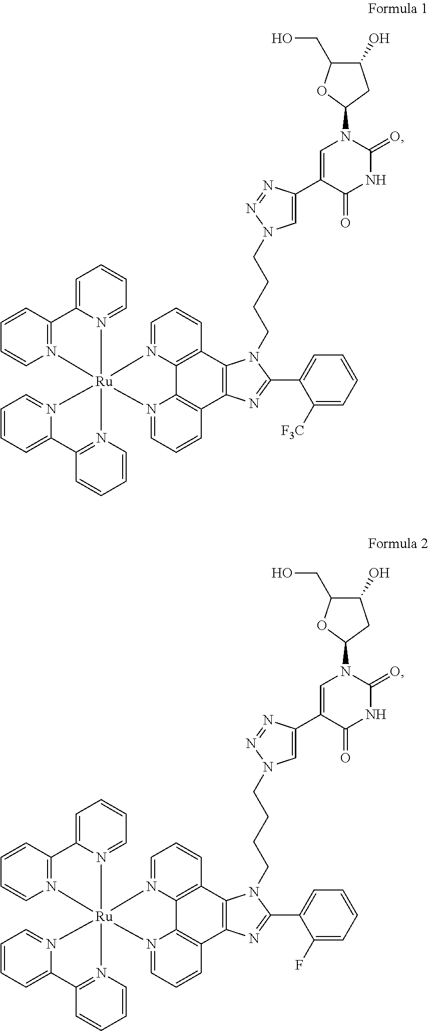

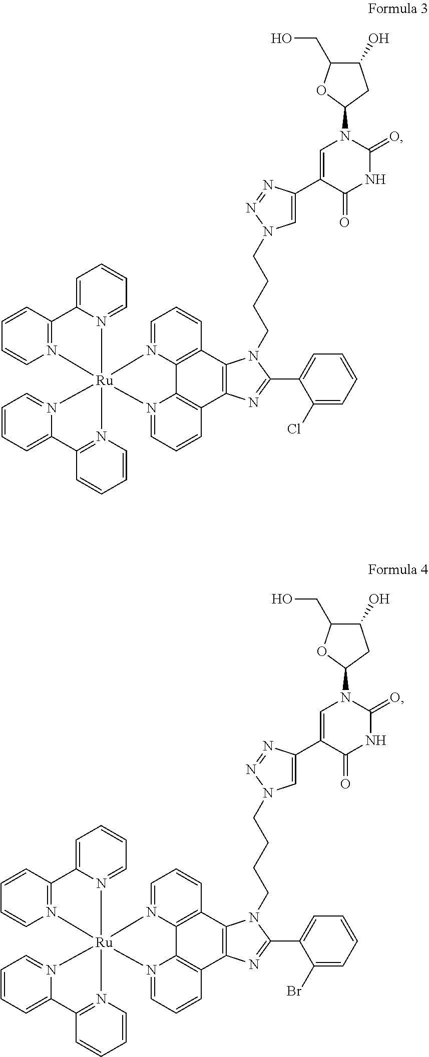

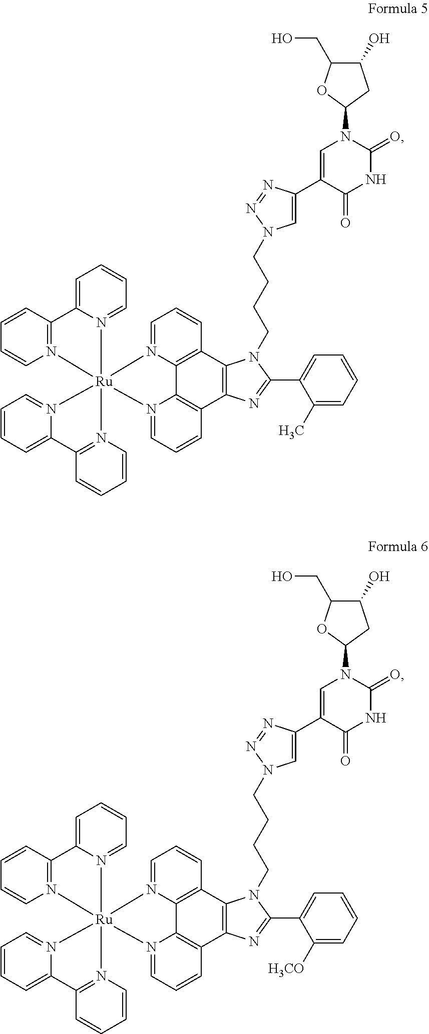

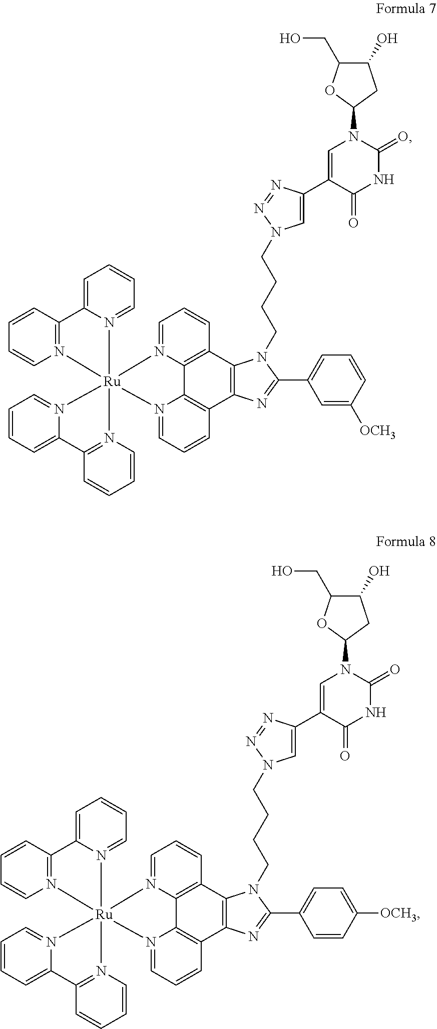

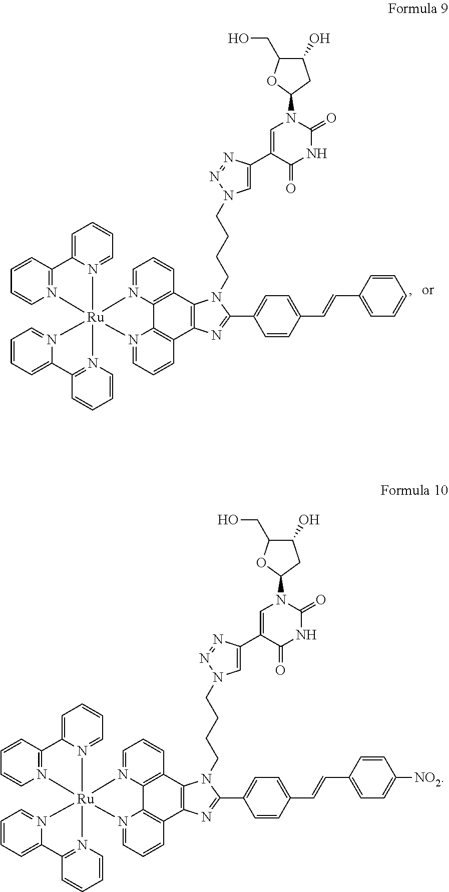

14. The method of claim 9, wherein the deoxynucleoside modified ruthenium complex has a structure represented by any one of Formulas 1 to 10:

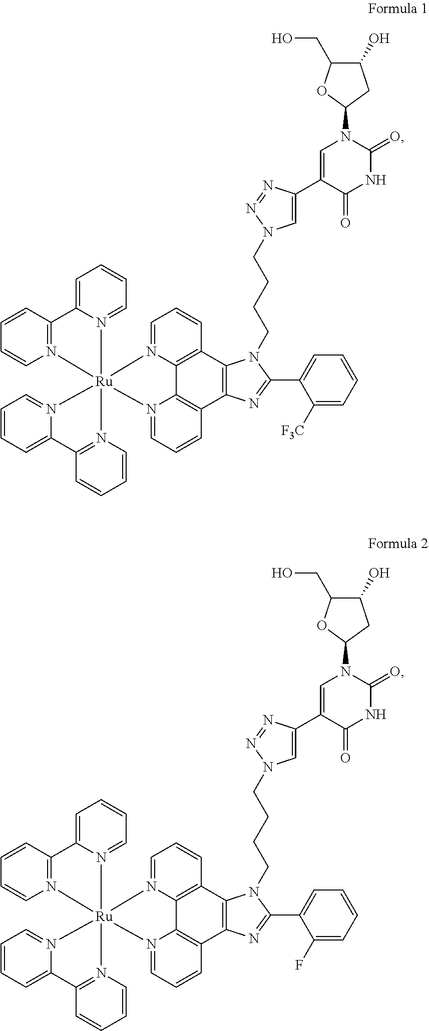

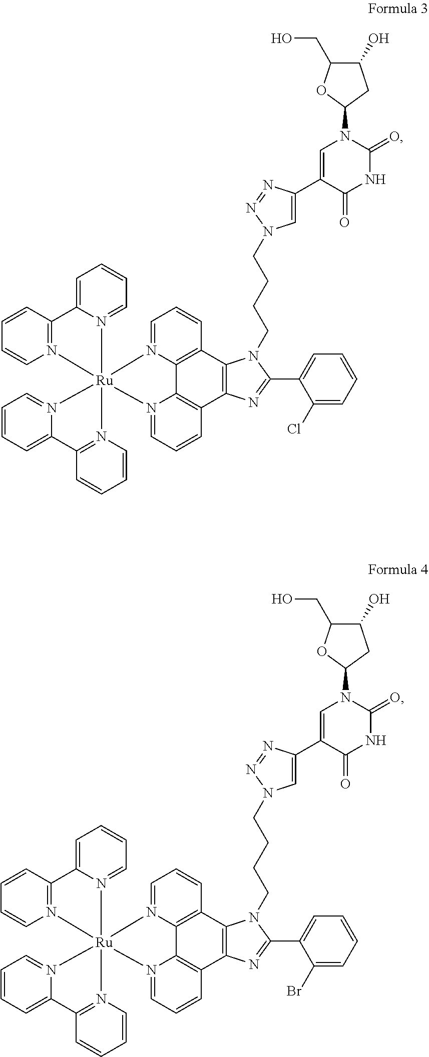

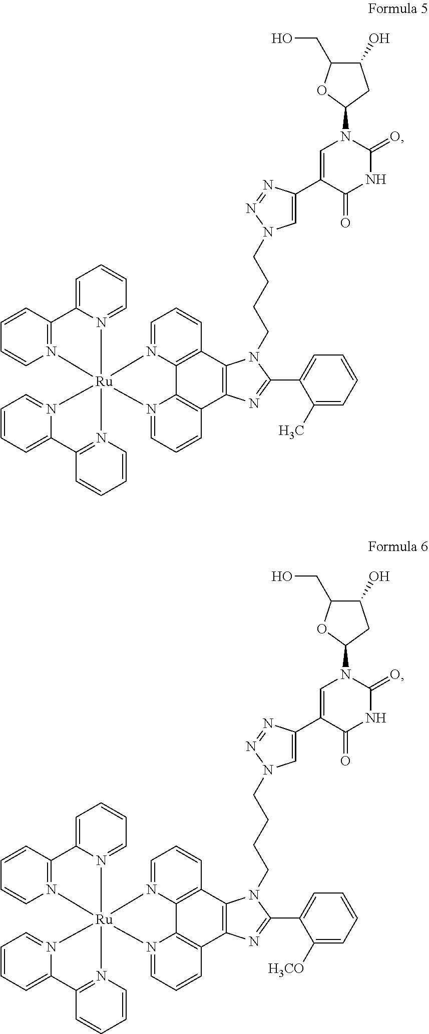

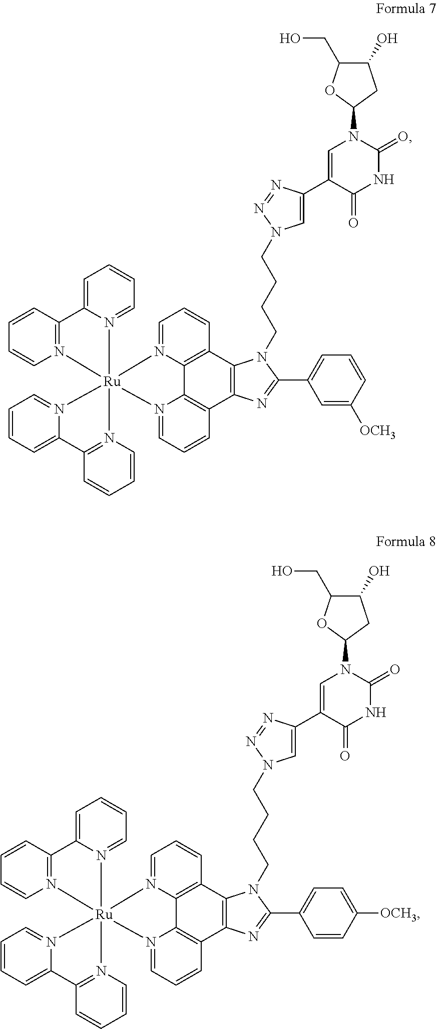

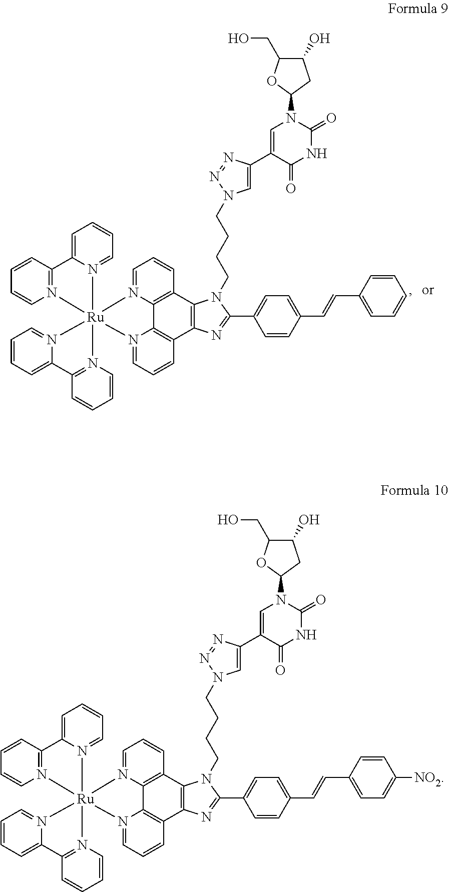

15. The method of claim 10, wherein the deoxynucleoside modified ruthenium complex has a structure represented by any one of Formulas 1 to 10: 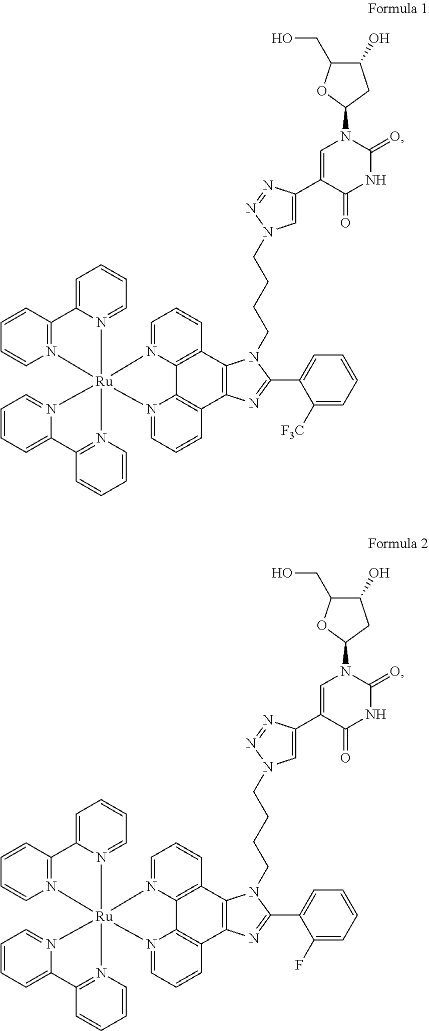 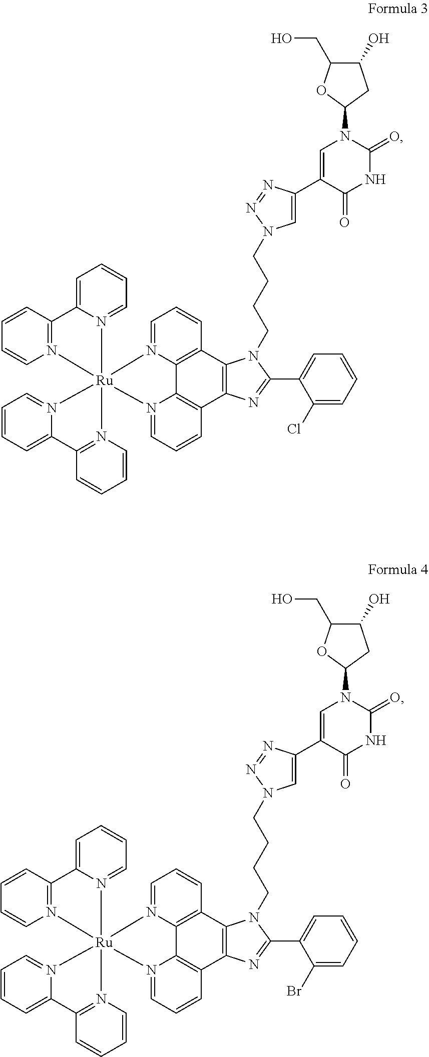 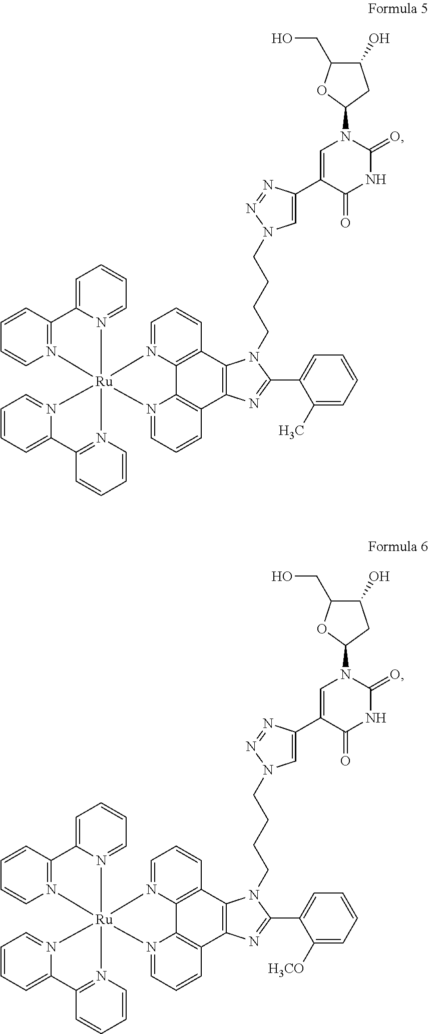 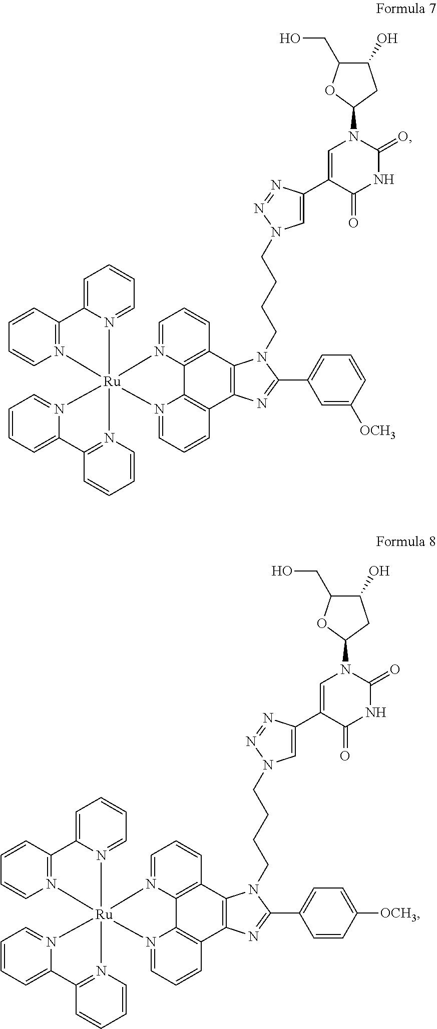 [chemical structure]
